SEQUENCE LISTING (12 sequences)

SEQ ID NO: 1 - DNA, 910 bp, Brassica oleracea
  CDS (46)..(726)
gaattcggcacgagaaaaagggaaagaaaaggaggaaccataaaaatggctgaggaacaacaaaaagcaggtcacagcaatggcgaaaacatcattcctcctgaagaagttgctaagttcctgcctgaaactgttgaggaaggtggatgggaaaaatgttgggaagacgggataacaccatgggaccaaggaagagccacacctctcgttgtccatcttgttgactcttcttctcttcctcttggccgtgctctagtccccggctgtggcggaggtcacgatgtggttgcgatggcaagccccgaacgtttcgttgttggtttggatatttccgaaagtgccctcgagaaagctgctgagacttacggctcctcaccgaaggccaagtactttacgttcgtgaaggaagacttcttcacatggcgtcctaacgaattatttgacctcatcttcgattatgtggtcttctgtgccattgagcctgagatgagacctgcatgggctaaatccatgtatgaactcttgaaacccgacggcgaactcataactctcatgtatccgattaccgatcatgatggtggacctccctacaaagtagctgtatctacctacgaagatgttttggttcccgtaggatttaaggcagtgtccatcgaggagaatccatactccattgccactcgtaagggcaaagagaagcttgggaggtggaagaagatcaattgatccaagaaagctctcttactcttcactaagagtattatatgatattctacttatctattaagaatgaattgtgttagtttactttatctgcattatataaaggttgtatttctgaatgtgatgattattcaacccttaataataaaataatgttttgtgatcaaaaaaaaaaaaaaaaaaaaaa

SEQ ID NO: 2 - protein, 226 aa, Brassica oleracea
MAEEQQKAGHSNGENIIPPEEVAKFLPETVEEGGWEKCWEDGITPWDQGRATPLVVHLVDSSSLPLGRALVPGCGGGHDVVAMASPERFVVGLDISESALEKAAETYGSSPKAKYFTFVKEDFFTWRPNELFDLIFDYVVFCAIEPEMRPAWAKSMYELLKPDGELITLMYPITDHDGGPPYKVAVSTYEDVLVPVGFKAVSIEENPYSIATRKGKEKLGRWKKIN

SEQ ID NO: 3 - DNA, 875 bp, Brassica oleracea
  CDS (52)..(732)
cacgagcaccaataaaaagaattattactagagcttgagagaaaccttaaaatggctgaggtacaacaaaactcaggtaacagcaatggtgaaaacatcatccctcccgaagatgttgctaagttcctgcccaaaactgttgatgaaggtggatgggaaaaatgttgggaagacggagtaacaccatgggaccaaggaagagccacacctctcgttgtccatcttgttgaatcttcttctcttcctcttggtcgtggtcttgtccccggctgtggcggaggtcacgatgtggttgcgatggcaagccccgaacgttacgttgttggtttggatatttctgaaagtgccctcgagaaagctgctgagacttacggctcctcaccgaaggccaagtactttacgttcgtgaaggaagacttcttcacatggcgtcctaacgaattattcgatctcattttcgattatgtggtcttctgtgccattgagccggagacgagacctgcatgggccaaagccatgtatgaactcttaaaacccgatggcgaactcattactctcatgtatccgattaccgatcatgatggtggaccgccatacaaagtagctgtctctacctacgaagatgttttggttccggtaggattcaaggcagtgtccattgaagagaatccatactccattgccactcgtaagggtaaagagaagcttgctaggtggaagaagatcaactgatccaagaaagatctctcttactctttactaagagtcttatatgcatattctaccaatgtattaagtatgaaatgaatattctacaaatttgccgattattaataataaaccttgatgtttgtgataaaaaaaaaaaaaaaaaa

SEQ ID NO: 4 - protein, 226 aa, Brassica oleracea
MAEVQQNSGNSNGENIIPPEDVAKFLPKTVDEGGWEKCWEDGVTPWDQGRATPLVVHLVESSSLPLGRGLVPGCGGGHDVVAMASPERYVVGLDISESALEKAAETYGSSPKAKYFTFVKEDFFTWRPNELFDLIFDYVVFCAIEPETRPAWAKAMYELLKPDGELITLMYPITDHDGGPPYKVAVSTYEDVLVPVGFKAVSIEENPYSIATRKGKEKLARWKKIN

SEQ ID NO: 5 - DNA, 1828 bp, Brassica oleracea
  CDS (1)..(97)
  CDS (281)..(411)
  CDS (562)..(648)
  CDS (727)..(827)
  CDS (903)..(1002)
  CDS (1133)..(1183)
  CDS (1790)..(1825)
atggctgaggaacaacaaaaagcaggtcacagcaatggcgaaaacatcattcctcctgaagaagttgctaagttcctgcctgaaactgttgaggaaggtgattcattcttctcttaatatttaccttatacaataaaagcgttttaggcctttgtctttactgatttatatggaaaaatcatggcggatttgtgacaaaaaaaagtcacactgatcatgatcatatgcttcttgattgaatctagtgaatacatcttccataactaagtaaaataaacaggtggatgggaaaaatgttgggaagacgggataacaccatgggaccaaggaagagccacacctctcgttgtccatcttgttgactcttcttctcttcctcttggccgtgctctagtccccggctgtggcggagttagtcttcttcttactcttcatatctctccctttttttctgttcaatttagaaaataaaatactgactgaaccaagatcgaagctagtttgaaatgttcataaggtaattaccccttttttgttgtaaaagataatgaatgtgagcagggtcacgatgtggttgcgatggcaagccccgaacgtttcgttgttggtttggatatttccgaaagtgccctcgagaaagctgctgagggtaatcattttctcactaagttacgttcgtcttcaattagtagaataaattattaataacctctggattacacatagacttacggctcctcaccgaaggccaagtactttacgttcgtgaaggaagacttcttcacatggcgtcctaacgaattatttgacctcatcttcgattatgtgtgagttaaccaactattttaacataatgttaaatataagtatttaatattccaattaactcatataattaacagggtcttctgtgccattgagcctgagatgagacctgcatgggctaaatccatgtatgaactcttgaaacccgacggcgaactcataactctcatgtatccggtaataattttcatttaacttttcgtgtggctattgaattatttaattacctatataatgcatcatgtatgccctataatcatagatcatgtgaattatgattaattagtttgcgttccttatttattagattaccgatcatgatggtggacctccctacaaagtagctgtatctacgtacgtaactattctcgttcttttttcttttttttttctgaaacacaatacgtaactattctgttagttattcttatatttatgactgatgaaaatctgtaattagcttaaatatttaactaaatatgttaattgtgcgtgcatttcttgaggtttagctacgaagatgttttggttcccgtaggatttaaggcagtgtccatcgaggagaatccatactccattgccactcgtaaggtacaaacacactgcctacattgtggaatggaatagtcaataatcatagaactgatacaattagatgtatatttgaaatcatataactaagttgtaattttttggctttgatgtatatacacatcccactgccattatatatagtacatgaatctagtacgtgaattatagtatagactatttttacatgataaaaaaaaacagaacaaaaattaccatctgtataagtcaaaatgttttagtatttttatgaaactattttcatataatactcaattaagtgactatattctagctgcattaattttaaagatacactaattgattactcattcgattgatatgatgtattttaacccaaaaaattgagcagggcaaagagaagcttgggaggtggaagaagatcaattga

SEQ ID NO: 6 - protein, 201 aa, Brassica oleracea
MAEEQQKAGHSNGENIIPPEEVAKFLPETVEEGGWEKCWEDGITPWDQGRATPLVVHLVDSSSLPLGRALVPGCGGGHDVVAMASPERFVVGLDISESALEKAAETYGSSPKAKYFTFVKEDFFTWRPNELFDLIFDYVVFCAIEPEMRPAWAKSMYELLKPDGELITLMYPITDHDGGPPYKVAVSTYGKEKLGRWKKIN

SEQ ID NO: 7 - DNA, 36 bp, Artificial sequence
  synthetic oligonucleotide
gagaggatccgtggctgaggaacaacaaaaagcagg

SEQ ID NO: 8 - DNA, 34 bp, Artificial sequence
  synthetic oligonucleotide
gagaggtacctcaattgatcttcttccacctccc

SEQ ID NO: 9 - DNA, 21 bp, Artificial sequence
  synthetic oligonucleotide
gtcatttcgaaccccagagtc

SEQ ID NO: 10 - DNA, 21 bp, Artificial sequence
  synthetic oligonucleotide
ctgaatgaactgcaggacgag

SEQ ID NO: 11 - DNA, 35 bp, Artificial sequence
  synthetic oligonucleotide
gagaggtaccatggctgaggaacaaaaaaagcagg

SEQ ID NO: 12 - DNA, 34 bp, Artificial sequence
  synthetic oligonucleotide
gagaggatcctcaattgatcttcttccacctccc

(19) United States
(12) Patent Application Publication
(10) Pub. No.: US 2007/0169214 A1
(43) Pub. Date: Jul. 19, 2007

(54) Thiol methyltransferase-based selection

(75) Inventors: Hargurdeep S. Saini, Waterloo (CA); Kumari Priyumvada Koonjul, Brossard (CA); Jihad Melhelm Attieh, El Koura (LB)

Correspondence Address: LADAS & PARRY LLP, 224 SOUTH MICHIGAN AVENUE, SUITE 1600, CHICAGO, IL, 60604, US

(73) Assignee: VALORISATION-RECHERCHE, SOCIETE EN COMMANDITE, QUEBEC (CA)

(21) Appl. No.: 10/592,567

(22) PCT Filed: Mar. 10, 2005
(86) PCT No.: PCT/CA05/00372

§ 371 Date: Sep. 12, 2006

Related U.S. Application Data
(60) Provisional application No. 60/552,214, filed on Mar. 12, 2004.

(51) Int. Cl. A01H 5/00; A01N 25/00; C12N 15/82; C12N 5/04

(52) U.S. Cl. 800/278; 530/808; 800/306; 435/419; 435/468; 504/116.1

(57) ABSTRACT

TMT-based methods for the selection of transgenic cells and the production of transgenic organisms are described herein. The toxin used in the selection methods is a TMT substrate. Also described herein are the recombinant cells, plants and progeny and seeds thereof having the TMT gene, a selectable marker system based on the TMT gene, and commercial packages relating to a TMT-based selectable marker system.

### CROSS REFERENCE TO RELATED APPLICATIONS

This application claims the benefit, under 35 U.S.C. § 119(e), of U.S. provisional patent application Ser. No. 60/552,214 filed Mar. 12, 2004, which is incorporated herein by reference in its entirety.

### FIELD OF THE INVENTION

The present invention relates to a selectable marker, and particularly relates to a thiol methyltransferase-based selectable marker, and uses thereof.

### BACKGROUND OF THE INVENTION

Genetic engineering has evolved rapidly in the recent years and is being applied to various fields. Despite many efforts towards increasing the efficiency of DNA uptake, introducing recombinant sequences into an organism remains a considerable challenge. In addition, many recombinant sequences do not confer a phenotype that can be screened easily.

In order to facilitate the development and maintenance of genetically modified organisms, genetic marker systems have been developed. The genetic marker is usually a known gene that confers, upon expression, a selectable trait. The genetic marker is co-introduced with the recombinant sequence of interest into a cell/organism. Organisms expressing the identifiable trait (genetic marker) are then considered also to have received the recombinant sequence. As such, recombinant organisms can easily be selected from a heterogeneous population of cells/organisms (i.e. a mixture of cells/organisms which contain or do not contain the recombinant sequence). In addition, genetic markers that provide a growth advantage to an organism can further be used to maintain a selective pressure on the recombinant organism. By enabling the discrimination of recombinant from non-recombinant organisms and maintaining a selective pressure on recombinant organisms, selectable markers play a pivotal role in genetic engineering methods.

Various categories of genetic markers exist. One category relates to markers that confer viability of an organism in the presence of a selective agent that would otherwise be lethal to that organism. In that particular case, the genetic marker usually confers resistance to a drug (e.g. antibiotic, a fungicide, etc.) or a toxin (e.g. pesticide, herbicide, etc.).

Another category relates to auxotrophy-based markers, where a host organism is genetically engineered to lack the ability to biosynthesize a component (e.g. by mutating the corresponding biosynthetic gene in its genome) necessary for viability (e.g. an amino acid), and must be maintained in media supplemented with the missing component. Selection is achieved by reintroducing the missing biosynthetic gene, thus allowing selectable growth on media lacking the component.

A further category relates to genetic markers which provide a distinguishing/detectable physical characteristic to the organism that expresses it. As such, recombinant organisms may be selected, for example, based on their ability to fluoresce (e.g. GFP system), or produce a detectable reaction product in the presence of a suitable substrate (e.g. luciferase or β-galactosidase system).

Although there are already a number of selectable markers available, there is a continuous need for other genetic markers. This is due to several reasons:
when recombinant organisms are being further transformed with an additional recombinant sequence, it is necessary to select for the newly formed recombinants with the help of an additional selectable marker;
known selectable markers are not conducive to selection in all organisms; it is therefore necessary to develop markers than can be used in certain organisms;
genetic engineering of the host cell genome must first be performed in some cases to allow for selection;
many of the compounds which have to be added to enable selection are antibiotics, and as such the use of such systems may lead to risk of spreading drug resistance in a natural environment;
some of the compounds which have to be added to enable selection are relatively costly.

### SUMMARY OF THE INVENTION

The invention relates to methods and systems for selecting a cell, tissue, organ and/or organism from a population thereof.

In a first aspect, the invention provides a method for selecting a first cell from a population of cells, said method comprising (a) providing said population of cells comprising said first cell, wherein said first cell comprises a first nucleic acid which encodes a polypeptide having TMT activity; (b) contacting said population of cells with a toxin, wherein said toxin is a TMT substrate; and (c) selecting said first cell by virtue of its increased tolerance to said toxin. In an embodiment, the first cell comprises a recombinant vector comprising the first nucleic acid. In a further embodiment, the method further comprises transforming a population of cells with the recombinant vector thereby to provide the population of cells comprising the first cell comprising the recombinant vector. In another embodiment, the toxin comprises a halide-containing compound, and further, the toxin comprises iodide. In another embodiment, the toxin comprises a thiol-containing compound or a thiolate-containing compound, and further, the toxin is selected from the group consisting of a thiocyanate, a sulfide and a thiol or thiolate. In another embodiment, the first cell is a prokaryotic cell, in a further embodiment, a bacterial cell (e.g. Escherichia coli).

In a further embodiment the LD₅₀ of said toxin is from about 150 to about 250 mM or from about 150 to about 200 mM. In yet another embodiment, the toxin is contacted with said population of cells at a toxin concentration from about 200 to about 250 mM, about 200 mM or about 250 mM.

In yet another embodiment, the first cell is an eukaryotic cell, in a further embodiment, a plant cell. The plant cell can be selected from the group consisting of a gymnosperm and an angiosperm plant cell. The angiosperm plant cell can be selected from the group consisting of a dicot and a monocot plant cell. In embodiments, the plant cell can be selected from the group consisting of a crop, horticultural, ornamental, fruit, forest tree and shrub plant cell. In embodiments, the plant cell can be selected from the group consisting of alfalfa, apple, apricot, Arabidopsis sp. (e.g. Arabidopsis thaliana), artichoke, arugula, asparagus, avocado, banana, barley, basil, beans (e.g. green bean), beet, blackberry, blueberry, broccoli, Brussels sprouts, cabbage, canola, cantaloupe, carrot, cassaya, castorbean, cauliflower, celery, cherry, chicory, cilantro, citrus, clementine, clover, coconut, coffee, corn, cotton, cranberry, cucumber, Douglas fir, eggplant, endive, escarole, eucalyptus, fennel, figs, garlic, gourd, grape, grapefruit, honey dew, jicama, kiwifruit, lettuce, leeks, lemon, lime, Loblolly pine, linseed, mango, Medicago sp., melon, nasturtium, nectarine, nut, oat, palm, rape, okra, olive, onion, orange, palm, papaya, parsley, parsnip, pea, peach, peanut, pear, pepper, persimmon, pine, pineapple, plantain, plum, pomegranate, poplar, potato, pumpkin, quince, radiata pine, radiscchio, radish, raspberry, rice, rye, sorghum, Southern pine, soybean, spinach, squash, strawberry, sugarbeet, sugarcane, sunflower, sweet potato, sweetgum, tangerine, tea, tobacco, tomato, triticale, turf, turnip, vine, watermelon, wheat, yam and zucchini plant cell. In an embodiment, the toxin is contacted with the cell at a concentration from about 100 μM to about 10 mM, in further embodiments, at a toxin concentration selected from the group consisting of about 100 μM, 125 μM, 1 mM, 2 mM, 2.5 mM, 4 mM, 5 mM, 7.5 mM and 10 mM. In another embodiment, the toxin is contacted with said population of cells at a toxin concentration of more than about 5 mM, 10 mM or 20 mM. In still another embodiment the eukaryotic cell is an animal cell, and further, a mammalian cell (e.g. human cell) or an immortalized cell. In still a further embodiment, the LD₅₀ of said toxin is from about 100 to about 150 mM, in a further embodiment, about 100 mM. In yet another embodiment, the toxin is contacted with said population of cells at a toxin concentration of greater than about 100 mM, 150 mM or 200 mM.

In an embodiment, the TMT polypeptide is a plant TMT, in a further embodiment, a Brassicaceae TMT, in yet a further embodiment, a Brassica TMT (e.g. Brassica oleracea TMT). In another embodiment, the Brassica oleracea TMT comprises a polypeptide having a sequence substantially identical to a sequence selected from the group consisting of SEQ ID NO: 2, SEQ ID NO: 4 and SEQ ID NO: 6. In an embodiment, the first nucleic acid comprises a nucleotide sequence substantially identical to a nucleic acid encoding a polypeptide having a sequence selected from the group consisting of SEQ ID NO: 2, SEQ ID NO: 4 and SEQ ID NO: 6, e.g. a nucleic acid having a sequence substantially identical to a sequence selected from the group consisting of SEQ ID NO: 1, SEQ ID NO: 3 and SEQ ID NO: 5. In addition, the vector may further comprise a second nucleic acid. In an embodiment first nucleic acid may be integrated into the genome of the first cell.

According to another aspect, the invention provides a method for selecting a first plant or part thereof from a population of plants or parts thereof, said method comprising (a) providing said population of plants or parts thereof comprising said first plant or part thereof, wherein said first plant or part thereof comprises a first nucleic acid which encodes a polypeptide having TMT activity; (b) contacting said population of plants or parts thereof with a toxin, wherein said toxin is a TMT substrate; and (c) selecting said first plant or part thereof by virtue of its increased tolerance to said toxin. In an embodiment, the first plant or part thereof comprises a recombinant vector comprising the first nucleic acid. In an embodiment, the part of a plant is selected from the group consisting of a tissue and an organ. In yet a further embodiment, the tissue is selected from the group consisting of ground tissue, dermal tissue, vascular tissue, parenchyma tissue and meristematic tissue. In still a further embodiment, the organ is selected from the group consisting of a root, leaf, stem, floral organ, embryo, endosperm, pollen grain, microspore, macrospore, gamete, zygote, seed, part of a seed and fruit. In an embodiment, the toxin is introduced into the soil or growth medium of said population of plants. The toxin can also be sprayed on plants or parts thereof.

In an embodiment, the toxin is contacted with the plants or parts thereof at a concentration from about 100 μM to about 10 mM, in further embodiments, at a toxin concentration selected from the group consisting of about 100 μM, 125 μM, 1 mM, 2 mM, 2.5 mM, 4 mM, 5 mM, 7.5 mM and 10 mm. In another embodiment, the toxin is contacted with said population of cells at a toxin concentration of more than about 5 mM, 10 mM or 20 mM.

In still another aspect, the invention provides a plant cell comprising a recombinant vector comprising a first nucleic acid which encodes a polypeptide having TMT activity. In an embodiment, the polypeptide is a plant TMT, in a further embodiment, a Brassicaceae TMT. In still a further embodiment, the Brassicaceae TMT is a Brassica TMT, in a further embodiment, a Brassica oleracea TMT. In yet another embodiment, the Brassica oleracea TMT comprises a polypeptide having a sequence substantially identical to a sequence selected from the group consisting of SEQ ID NO: 2, SEQ ID NO: 4 and SEQ ID NO: 6. In embodiments, the plant cell is selected from the group consisting of a gymnosperm and an angiosperm plant cell. In an embodiment, the angiosperm plant cell is selected from the group consisting of a dicot and a monocot plant cell. In another embodiment, the plant cell is selected from the group consisting of a crop, horticultural, ornamental, fruit, forest tree and shrub plant cell. In still another embodiment, the plant cell is selected from the group consisting of Medicago sp., Arabidopsis sp., tobacco, potato, rice, tomato, pepper, wheat, corn, canola and bean cell. In still another embodiment, the first nucleic acid comprises a nucleotide sequence substantially identical to a sequence selected from the group consisting of SEQ ID NO: 1, SEQ ID NO: 3 and SEQ ID NO: 5. In an embodiment, the vector may further comprise a second nucleic acid. The second nucleic acid may be, for example, a heterologous nucleic acid such as a gene or sequence of interest to be introduced into the cell. The first nucleic acid can also be integrated into the genome of the plant cell.

In a further aspect, the invention provides a transgenic plant comprising the plant cell described above, or a progeny, tissue, organ or seed thereof.

In still another aspect, the invention also provides a method of preparing the transgenic plant described herein, said method comprising regenerating the transgenic plant from the plant cell described herein.

In yet another aspect, the invention provides a selectable marker system for the selection of a first cell from a population of cells comprising (a) a recombinant vector comprising a first nucleic acid which encodes a polypeptide having TMT activity; and (b) a toxin, wherein said toxin is a TMT substrate. In an embodiment, the vector further comprises a unique restriction endonuclease cleavage site. In another embodiment, the tolerance of said first cell to said toxin is increased when said vector is introduced into said first cell. In yet another embodiment, the polypeptide having TMT activity comprises a sequence substantially identical to a sequence selected from the group consisting of SEQ ID NO: 2, SEQ ID NO: 4 and SEQ ID NO: 6.

In still another aspect, the invention provides a commercial package comprising (a) a recombinant vector comprising a nucleic acid encoding a polypeptide having TMT activity; and (b) instructions for the selection of a first cell from a population of cells. In an embodiment, the instructions set forth a method comprising (a) contacting said vector with said population of cells under conditions that said vector will be introduced into said first cell; (b) contacting said population of cells with a toxin, wherein said toxin is a TMT substrate; and (c) selecting said first cell by virtue of its increased tolerance to said toxin. In an embodiment, the commercial package further comprises a toxin, wherein said toxin is a TMT substrate. In another embodiment, the vector further comprises a unique restriction endonuclease cleavage site. In a further embodiment, the first cell is a transgenic plant cell. In still a further embodiment, the polypeptide has a sequence substantially identical to a sequence selected from the group consisting of SEQ ID NO: 2, SEQ ID NO: 4 and SEQ ID NO: 6.

### BRIEF DESCRIPTION OF THE DRAWINGS

FIG. 1. Bacterial growth measured by optical density at a wavelength of 600 nm (OD₆₀₀) of cultures of transgenic (right panel) and non-transgenic (left panel) bacteria in the presence of increasing concentrations of thiocyanate (⁻SCN) supplied as NaSCN. SCN concentration is expressed in mM.

FIG. 2. Bacterial growth measured by optical density at a wavelength of 600 nm (OD₆₀₀) of cultures of transgenic (right panel) and non-transgenic (left panel) bacteria in the presence of increasing concentrations of iodide (I⁻) supplied as KI. I⁻ concentration is expressed in mM.

FIG. 3. Growth analysis of non-transgenic (−TMT, clone 10.3-35, panels A, C, E, G, I, K, M, 0 and Q) vs transgenic (+TMT, clone 31.1#7.4-88, panels B, D, F, H, J, L, N, P and R) roots in the presence of various concentrations of ⁻SCN. A and B, 0 mM ⁻SCN; C to F, 2 mM ⁻SCN; G to J, 5 mM ⁻SCN; K to N, 10 nm ⁻SCN; 0 to R, 20 mM ⁻SCN. E and F represent a 2× enlargement of the roots shown in C and D respectively. I and J represent a 2× enlargement of the roots shown in G and H respectively. M and N represent a 2× enlargement of the roots shown in K and L respectively. Q and R represent a 2× enlargement of the roots shown in 0 and P respectively.

FIG. 4. A. Graphical representation of total root length of potato roots as measured by WinRhizo™ software with respect to ⁻SCN concentration (mM). Each line represents a different clone. Solid lines represent all transgenic roots while the broken lines represent the non-transgenic roots. B. Enlarged version of FIG. 4A, showing the effect at the lower concentrations of ⁻SCN (0-10 mM).

FIG. 5. Immunofluorescent detection of GFP expression in non-transgenic (A to C and G to I) and transgenic (D to F and J to L) HEK mammalian cells in the presence of various concentrations of ⁻SCN (A and D, 0 mM ⁻SCN; B and E, 20 mM SCN; C and F, 50 mM SCN; G and J, 100 mM ⁻SCN; H and K, 150 mM ⁻SCN; I and L, 200 mM SCN).

FIG. 6. Immunofluorescent detection of GFP expression in non-transgenic (A to C and G to I) and transgenic (D to F and J to L) HEK mammalian cells in the presence of various concentrations of KI (A and D, 0 mM KI; B and E, 20 mM KI; C and F, 50 mM KI; G and J, 100 mM KI; H and K, 150 mM KI; I and L, 200 mM KI).

FIG. 7. Total TMT activity (nmol/min) measured with respect to increasing concentration of the toxin (mM of ⁻SCN or I⁻) in cells transfected with 400 or 200 ng of DNA.

FIG. 8. Brassica oleracea thiol methyltransferase 1 (TMT1) cDNA sequence (SEQ ID NO: 1, GenBank accession number AF387791) and corresponding polypeptide sequence (SEQ ID NO: 2, GenBank accession number AAK69760).

FIG. 9. Brassica oleracea thiol methyltransferase 2 (TMT2) cDNA sequence (SEQ ID NO: 3, GenBank accession number AF387792) and corresponding polypeptide sequence (SEQ ID NO: 4, GenBank accession number AAK69761).

FIG. 10. Brassica oleracea thiol methyltransferase 1 (TMT1) genomic DNA sequence (SEQ ID NO: 5, Genbank accession number AF387793) and corresponding polypeptide sequence (SEQ ID NO: 6, GenBank accession number AAK69762).

FIG. 11. Visual selection of sunflower root clones. Root clones were allowed to grow for 7 days on MS/phytagel before their length was measured using Winrhizo™. The Petri dish shown contains 10 mM SCN and the roots are the individual sunflower clones randomly chosen for assay.

FIG. 12. Effect of 10 mM SCN on sunflower root clones length (cm) (left Y-axis) and in vivo TMT activity (right Y-axis).

FIG. 13. PCR analysis of sunflower clones (TMT-specific primers).

FIG. 14. Visual selection of tobacco root clones. Root clones were allowed to grow for 7 days on MS/phytagel before their length was measured using Winrhizo™. The Petri dish shown contains 4 mM SCN and the roots are the individual tobacco clones randomly chosen for assay.

FIG. 15. Effect of 4 mM SCN on tobacco root clones length (cm) (left Y-axis) and in vivo TMT activity (right Y-axis).

FIG. 16. PCR analysis of tobacco clones (TMT-specific primers).

FIG. 17. Visual selection of carrot root clones. Root clones were allowed to grow for 7 days on MS/phytagel before their length was measured using Winrhizo™. The Petri dish shown contains 125 μM SCN and the roots are the individual carrot clones randomly chosen for assay.

FIG. 18. Effect of 125 μM SCN on carrot root clones length (cm) (left Y-axis) and in vivo TMT activity (right Y-axis).

FIG. 19. PCR analysis of carrot clones (TMT-specific primers).

FIG. 20. Effect of 5 mM SCN on nasturtium root clones length (cm) (left Y-axis) and in vivo TMT activity (right Y-axis).

FIG. 21. PCR analysis of nasturtium clones (TMT-specific primers).

FIG. 22. Effect of 100 μM SCN on basil root clones length (cm) (left Y-axis) and in vivo TMT activity (right Y-axis).

FIG. 23. PCR analysis of basil root clones (TMT-specific primers).

FIG. 24. Effect of 5 mM SCN on potato root clones length (cm) (left Y-axis) and in vivo TMT activity (right Y-axis).

FIG. 25. PCR analysis of potato root clones (TMT-specific primers).

FIG. 26. Visual selection of green bean root clones. Root clones were allowed to grow for 7 days on MS/phytagel before their length was measured using Winrhizo™. The Petri dish shown contains 7.5 mM SCN and the roots are the individual green bean clones randomly chosen for assay.

FIG. 27. Effect of 7.5 mM SCN on green bean root clones length (cm) (left Y-axis) and in vivo TMT activity (right Y-axis).

FIG. 28. PCR analysis of green bean root clones (TMT-specific primers).

FIG. 29. Visual selection of tomato root clones. Root clones were allowed to grow for 7 days on MS/phytagel before their length was measured using Winrhizo™. The Petri dish shown contains 7.5 mM SCN and the roots are the individual green bean clones randomly chosen for assay.

FIG. 30. Effect of 7.5 mM SCN on tomato root clones length (cm) (left Y-axis) and in vivo TMT activity (right Y-axis).

FIG. 31. PCR analysis of tomato root clones (TMT-specific primers).

FIG. 32. TMT as a selectable marker in whole dicot plants. Seeds of wild type (WT) and TMT transgenic (T) tobacco plants (produced by transformation using conventional A. tumefaciens infection and selected with kanamycin) were sown in MS/phytagel containing different SCN concentrations. Pictures were taken 21 days after germination.

FIG. 33. TMT as a selectable marker in whole dicot plants (higher magnification of FIG. 32). Seeds of wild type (WT) and TMT transgenic (T) tobacco plants (produced by transformation using conventional A. tumefaciens infection and selected with kanamycin) were sown in MS/phytagel containing different SCN concentrations. Pictures were taken 21 days after germination.

FIG. 34. Effect of SCN on in vivo TMT activity of transgenic/non-transgenic tobacco plants.

FIG. 35. TMT as a selectable marker in whole dicot plants. Wild type (TT) (naturally contains TMT), heterozygous (Tt) and double recessive TMT knock out A. thaliana plants were sown in MS/phytagel containing different SCN concentrations. Pictures were taken 21 days after germination.

FIG. 36. Effect of SCN on TMT activity in 3 Arabidopsis phenotypes.

### DETAILED DESCRIPTION OF THE INVENTION

Described herein are results demonstrating the use of TMT activity as a selectable marker.

In a first embodiment, a method is herein described for the selection of a first cell from a population of cells by exposing said population of cells to a toxin (e.g. a TMT substrate). The first cell comprises a nucleic acid encoding a polypeptide having TMT activity, e.g. a TMT gene. The first cell is more tolerant to the toxin than a second cell that lacks said TMT activity.

In an embodiment, an isolated nucleic acid, for example a nucleic acid sequence encoding a polypeptide having TMT activity, or an active homologue, fragment or variant thereof, is incorporated into a recombinant expression vector. A “vector” as described herein refers to a vehicle that carries a nucleic acid sequence and serves to introduce the nucleic acid sequence into a host cell. In an embodiment, the vector will comprise transcriptional regulatory sequences or a promoter operably-linked to a nucleic acid comprising a sequence capable of encoding a polypeptide having TMT activity. A first nucleic acid sequence is “operably-linked” with a second nucleic acid sequence when the first nucleic acid sequence is placed in a functional relationship with the second nucleic acid sequence. For instance, a promoter is operably-linked to a coding sequence if the promoter affects the transcription or expression of the coding sequences. Generally, operably-linked DNA sequences are contiguous and, where necessary to join two protein coding regions, in reading frame. However, since, for example, enhancers generally function when separated from the promoters by several kilobases and intronic sequences may be of variable lengths, some polynucleotide elements may be operably-linked but not contiguous. “Transcriptional regulatory element” is a generic term that refers to DNA sequences, such as initiation and termination signals, enhancers, and promoters, splicing signals, polyadenylation signals which induce or control transcription of protein coding sequences with which they are operably-linked.

A promoter can be used to express, in a constitutive manner, a nucleic acid of interest, such as a nucleic acid encoding a polypeptide having TMT activity and/or a second nucleic acid. On the other hand, a promoter can also be used to express the nucleic acid of interest in a specific manner (e.g. tissue- or temporal-specific). For example, a promoter can be selected for its ability to express a nucleic acid of interest in specific areas of a cell (e.g. organelles, nucleus, cytoplasm, etc.), or an organ (e.g. seed, leaf, fruit, flower, skin, bone, immune cells, heart, etc.), during specific developmental stages of an organism (cell cycle, life cycle, hormonal cycle, etc.) or in response to a specific stimulus or inducer (e.g. an inducible promoter).

Depending on the promoter used and the type of host cell, the level of expression of the nucleic acid of interest may vary. In an embodiment, the promoter may allow high level of expression (i.e. higher than the average level of expression by endogenous promoters in a particular organism) of the nucleic acid of interest. In another embodiment, the promoter may permit a lower level of expression of the nucleic acid of interest. Such promoters conferring varying levels of expression are known for a number of systems.

One skilled in the art will appreciate that many recombinant expression vectors can be used to carry a nucleic acid of the invention. A “plasmid”, which is usually defined as an autonomously, self-replicating DNA molecule, can be used as a vector. Depending on the nature of the vector, more than one copy of the plasmid may be present in the host cell. Many different plasmids have been developed from modified viral genomes (e.g. cosmid, lentiviral-based, adenoviral-based, etc.) and engineered existing chromosomes (e.g. yeast and human artificial chromosomes). On the other hand, some vectors cannot autonomously replicate. These vectors are usually integrated into the host cell's genome. Vector integration can either cause expression of the nucleic acid they carry (e.g. transgenic plant or animal) or disruption of the coding sequence at the site of integration (e.g. genetically disrupted or “knockout” plant or animal). Depending on the intended use, vectors can also be single or double-stranded or a combination of both. Vectors can also be linear or circular.

The recombinant expression vector of the present invention can be constructed by standard techniques known to one of ordinary skill in the art and found, for example, in Sambrook and Russell (2001) Molecular Cloning: A Laboratory Manual. (3rd edition, 2001, Cold Spring Harbor Laboratory Press, New York). A variety of strategies are available for ligating fragments of DNA, the choice of which depends on the nature of the termini of the DNA fragments and can be readily determined by persons skilled in the art. The vectors of the present invention may also contain other sequence elements to facilitate vector propagation and selection in bacteria and host cells. In addition, the vectors of the present invention may comprise a sequence of nucleotides for one or more restriction endonuclease sites (e.g. within a region sometimes referred to in the art as a “polylinker”). Coding sequences such as for other selectable markers and reporter genes are well known to persons skilled in the art.

A recombinant expression vector comprising a nucleic acid sequence of the present invention may be introduced into a host cell, which may include a living cell capable of expressing the protein-coding region from the defined recombinant expression vector. In embodiments, the living cell may be found in culture or within a living organism. Accordingly, the invention also provides host cells containing the recombinant expression vectors of the invention. The terms “host cell”, “recombinant host cell” and “transgenic cell” are used interchangeably herein. Such terms refer not only to the particular subject cell but also to the progeny or potential progeny of such a cell. Because certain modifications may occur in succeeding generations due to either mutation or environmental influences, such progeny may not, in fact, be identical to the parent cell, but are still included within the scope of the term as used herein.

In an embodiment, the vector described herein comprise a first nucleic acid encoding a polypeptide having TMT activity and a second nucleic acid. The second nucleic acid may be, for example, any nucleic acid (e.g. a gene) of interest for introduction into or expression in the cell or plant, such as a heterologous nucleic acid.

In an embodiment, the host cell is a prokaryotic cell, such as a bacterial cell. In an embodiment, the bacterial host cell is Escherichia coli. In further embodiments, other prokaryotic cells can be used, including, but not limited to, gram-positive and gram negative bacteria, coccus, bacillus, spirillum, aerobic and/or anaerobic organisms, spores, mycoplasma, etc.

In an embodiment, the host cell can be an eukaryotic host cell, in a further embodiment, a plant cell. The plant cell can optionally be a gymnosperm or an angiosperm plant cell. As used herein, a gymnosperm is defined as a non-flowering seed plant. The most familiar members of this category include, but are not limited to conifers, such as pine, spruce, cedar or fir. The term “angiosperm” refers to a flowering plant whose seeds are enclosed in ovaries, which are later referred to as fruits when they mature. Virtually all crops and ornamentals fall into the angiosperm category (e.g. wheat, rice, Medicago, canola, Arabidopsis, cabbage, petunia, carrot, potato, tomato, etc.). Angiosperms are further divided into two subgroups: monocots and dicots. Monocots, as used herein, are flowering plants with one cotyledon in the seed. Examples of monocots include, but are not limited to grasses (e.g. wheat, rice, corn, barley, rye), orchids, lilies, palms and onions. On the other hand, dicots are defined as flowering plants with two cotyledons in the seed. Examples of dicots include, but are not limited to, bean, peas, sunflower, tomato, roses, oak tree, mango, Medicago sp., Arabidopsis sp., canola and cabbage. In embodiments, the plant cell includes, but is not limited to a dicot, monocot, crop, horticultural, ornamental, fruit, forest tree and shrub plant cell. In further embodiments, plant cells also include alfalfa (Medicago sp.), apple, apricot, Arabidopsis sp. (e.g. Arabidopsis thaliana), artichoke, arugula, asparagus, avocado, banana, barley, basil, beans (e.g. green bean), beet, blackberry, blueberry, broccoli, Brussels sprouts, cabbage, canola, cantaloupe, carrot, cassaya, castorbean, cauliflower, celery, cherry, chicory, cilantro, citrus, clementine, clover, coconut, coffee, corn, cotton, cranberry, cucumber, Douglas fir, eggplant, endive, escarole, eucalyptus, fennel, figs, garlic, gourd, grape, grapefruit, honey dew, jicama, kiwifruit, lettuce, leeks, lemon, lime, Loblolly pine, linseed, mango, Medicago sp., melon, nasturtium, nectarine, nut, oat, palm, rape, okra, olive, onion, orange, palm, papaya, parsley, parsnip, pea, peach, peanut, pear, pepper, persimmon, pine, pineapple, plantain, plum, pomegranate, poplar, potato, pumpkin, quince, radiata pine, radiscchio, radish, raspberry, rice, rye, sorghum, Southern pine, soybean, spinach, squash, strawberry, sugarbeet, sugarcane, sunflower, sweet potato, sweetgum, tangerine, tea, tobacco, tomato, triticale, turf, turnip, vine, watermelon, wheat, yam and zucchini plant cell.

In embodiments the host plant cell can be cultured in vitro or grown in vivo. When a plant cell is grown in vivo, it may be present in a plant or part thereof, such as a tissue (e.g. ground tissue, dermal tissue, vascular tissue, parenchyma tissue or meristematic tissue) or organ (e.g. root, leave, stem, floral organ, embryo, endosperm, pollen grain, microspore, macrospore, gamete, zygote, seed, part of a seed or a fruit) or a plant. Alternatively, the plant cell can be used to generate a plant, parts of a plant, plant tissues and organs. Consequently, the cells of the regenerated plant, parts of the plant, tissues and organs will comprise the coding sequence for the polypeptide with TMT activity.

Accordingly the presence of said host plant cell having a coding sequence for a polypeptide with TMT activity in a plant or part thereof may be used to select the plant or part thereof from a population of plants or parts thereof. As such, the toxin can optionally be applied on or contacted with the plant cells having the polypeptide with TMT activity for the selection of those cells from a population of cells. The selected cells can then further be grown (in vivo or in vitro) in the presence or absence of the toxin to generate other plant cells, plant parts, tissues and organs. In a further embodiment, plant parts or plants are first regenerated from plant cells transformed with a vector comprising the coding sequence for a polypeptide having TMT activity, before being contacted with the toxin. The regenerated plant parts and plants can then be screened for the presence or absence of TMT activity by applying the toxin on the regenerated plant parts or plants. Once this selection has been achieved, the selective pressure (i.e. contact with the toxin) can either be maintained on the regenerated parts or plants or removed.

In a further embodiment, the toxin can also be directly applied (e.g. sprayed) on plants or plant parts.

In a further embodiment, the toxin is applied to the soil or growth medium or matrix of said plant or part thereof. For example, the toxin may be applied as an additive to the soil or growth medium/matrix, in conjunction or not with other additives, in embodiments during watering or administration of nutrients to the plant, or may be added to the soil or growth medium prior to the planting of the plant therein.

In yet another embodiment, the selected plant or plant parts can further be used to produce progeny, tissue, organ or seed. The production of progeny, tissue, organ or seed can be performed under selective pressure (i.e. in the presence of the toxin). Alternatively, the production of progeny, tissue, organ or seed can be performed in the absence of selective pressure, in which case the toxin can optionally be applied to the progeny, tissue, organ or seed once they have been produced to assay for the presence of the selectable marker (i.e. TMT activity).

In further embodiments the eukaryotic cell can be a non-plant cell (e.g. yeast and fungus, animal, invertebrate, etc.). In a further embodiment, the eukaryotic host cell is an animal cell (e.g. mammalian cell). In an embodiment, the animal host cell can be cultured in vitro as a monolayer culture, a suspended cell culture, a tissue-like culture or an organ-like culture. The animal cell can also be used to generate a transgenic organism.

In a further embodiment, the eukaryotic host cell can also be an immortalized cell. The immortalized cell can be cultured in vitro to form a monolayer culture, a suspended cell culture or can be seeded on beads or other matrix for a continuous or batch cell culture. The immortalized cells can also be cultured in vivo (e.g. production of ascites fluid in the mouse intraperitoneal cavity).

Vector DNA can be introduced into cells via conventional transformation or transfection techniques. The terms “transformation” (or “transforming”) and “transfection” (or “transfecting”) refer to techniques for introducing foreign nucleic acid into a host cell, including calcium phosphate or calcium chloride co-precipitation, DEAE-dextran-mediated transfection, lipofection, electroporation, microinjection, bacterial-mediated and viral-mediated transfection. Suitable methods for transforming or transfecting host cells can for example be found in Sambrook and Russell (2001) Molecular Cloning: A Laboratory Manual, and other laboratory manuals. Methods for introducing DNA into plant cells are also known to those skilled in the art (e.g. bombardment, bacterial-mediated (e.g. Agrobacterium tumefaciens and Agrobacterium rhizogenes) transformation, protoplast transformation, gene transfer into pollen, and injection into immature embryos or reproductive organs).

As used herein, “TMT activity” relates to a methylation reaction of a suitable substrate (i.e. a TMT substrate—see below), such as a bisulfide (e.g. HS⁻), halide (e.g. I⁻, Br⁻ or Cl⁻) and a thiol or thiolate (e.g. ⁻SCN). Methods for determining TMT activity include for example the method described in Examples 2 and 4 below and in Attieh et al. ([2000] Plant, Cell and Environment 23: 165-174). TMT activity has been observed in different plant species such as Brassica sp., Iberis sp., Armorcia sp., Barbara sp, Cackile sp., Crambe sp., Reseda sp., Eruca sp., Isatis sp., Raphanus sp., Arabidopsis sp. or Tropaeolum sp. “TMT gene” refers to a DNA sequence that encodes a polypeptide having such thiol methyltransferase activity. Many different organisms possess such TMT genes. In an embodiment, the TMT gene is a plant TMT gene (e.g. cabbage, kale, swede, cauliflower, Brussels sprout, kohlrabi, broccoli, turnip, radish, mustard or nasturtium). In a further embodiment, the TMT gene encodes a Brassicaceae TMT polypeptide, and further, a Brassica oleracea TMT polypeptide (e.g. SEQ ID NO: 2; SEQ ID NO: 4; SEQ ID NO: 6).

A homologue, variant and/or fragment of a TMT which retains TMT activity may also be used in the methods of the invention. Homologue include protein sequences which are substantially identical to polypeptide sequence of a TMT (e.g. SEQ ID Nos: 2, 4 or 6), sharing significant structural and functional homology with a TMT. Variants include, but are not limited to, proteins or peptides which differ from a TMT by any modifications, and/or amino acid substitutions, deletions or additions. Modifications can occur anywhere including the polypeptide backbone, (i.e. the amino acid sequence), the amino acid side chains and the amino or carboxy termini. Such substitutions, deletions or additions may involve one or more amino acids. Fragments include a fragment or a portion of a TMT or a fragment or a portion of a homologue or variant of a TMT. Similarly, such homologues, variants and/or fragments include polypeptides or proteins encoded by a nucleic acid sequence which is substantially identical to, or is related by hybridization criteria (see below) to a nucleic acid sequence capable of encoding a TMT (such as those having a polypeptide sequence of SEQ ID NOs: 2, 4 or 6), such as the TMT DNA sequences set forth in SEQ ID NOs: 1, 3 or 5.

In an embodiment, the TMT gene encodes a polypeptide whose sequence is substantially identical to a TMT polypeptide, e.g. substantially identical to a sequence selected from the group consisting of SEQ ID NO: 2, SEQ ID NO: 4 and SEQ ID NO: 6. “Homology” and “homologous” refers to sequence similarity between two peptides or two nucleic acid molecules. Homology can be determined by comparing each position in the aligned sequences. A degree of homology between nucleic acid or between amino acid sequences is a function of the number of identical or matching nucleotides or amino acids at positions shared by the sequences. As the term is used herein, a nucleic acid sequence is “homologous” to another sequence if the two sequences are substantially identical and the functional activity of the sequences is conserved (as used herein, the term “homologous” does not infer evolutionary relatedness). Two nucleic acid sequences are considered “substantially identical” if, when optimally aligned (with gaps permitted), they share at least about 50% sequence similarity or identity, or if the sequences share defined functional motifs. In alternative embodiments, sequence similarity in optimally aligned substantially identical sequences may be at least 60%, 70%, 75%, 80%, 85%, 90% or 95%. As used herein, a given percentage of homology between sequences denotes the degree of sequence identity in optimally aligned sequences. An “unrelated” or “non-homologous” sequence shares less than 40% identity, though preferably less than about 25% identity, with any of SEQ ID NOs 1-6.

Substantially complementary nucleic acids are nucleic acids in which the complement of one molecule is substantially identical to the other molecule. Two nucleic acid or protein sequences are considered substantially identical if, when optimally aligned, they share at least about 70% sequence identity. In alternative embodiments, sequence identity may for example be at least 75%, at least 80%, at least 85%, at least 90%, or at least 95%. Optimal alignment of sequences for comparisons of identity may be conducted using a variety of algorithms, such as the local homology algorithm of Smith and Waterman, 1981, Adv. Appl. Math 2: 482, the homology alignment algorithm of Needleman and Wunsch, 1970, J. Mol. Biol. 48:443, the search for similarity method of Pearson and Lipman, 1988, Proc. Natl. Acad. Sci. USA 85: 2444, and the computerised implementations of these algorithms (such as GAP, BESTFIT, FASTA and TFASTA in the Wisconsin Genetics Software Package, Genetics Computer Group, Madison, Wis., U.S.A.). Sequence identity may also be determined using the BLAST algorithm, described in Altschul et al., 1990, J. Mol. Biol. 215:403-10 (using the published default settings). Software for performing BLAST analysis may be available through the National Center for Biotechnology Information (through the internet at http://www.ncbi.nlm.nih.gov/). The BLAST algorithm involves first identifying high scoring sequence pairs (HSPs) by identifying short words of length W in the query sequence that either match or satisfy some positive-valued threshold score T when aligned with a word of the same length in a database sequence. T is referred to as the neighborhood word score threshold. Initial neighborhood word hits act as seeds for initiating searches to find longer HSPS. The word hits are extended in both directions along each sequence for as far as the cumulative alignment score can be increased. Extension of the word hits in each direction is halted when the following parameters are met: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T and X determine the sensitivity and speed of the alignment. The BLAST program may use as defaults a word length (W) of 11, the BLOSUM62 scoring matrix (Henikoff and Henikoff, 1992, Proc. Natl. Acad. Sci. USA 89: 10915-10919) alignments (B) of 50, expectation (E) of 10 (or 1 or 0.1 or 0.01 or 0.001 or 0.0001), M=5, N=4, and a comparison of both strands. One measure of the statistical similarity between two sequences using the BLAST algorithm is the smallest sum probability (P(N)), which provides an indication of the probability by which a match between two nucleotide or amino acid sequences would occur by chance. In alternative embodiments of the invention, nucleotide or amino acid sequences are considered substantially identical if the smallest sum probability in a comparison of the test sequences is less than about 1, preferably less than about 0.1, more preferably less than about 0.01, and most preferably less than about 0.001.

An alternative indication that two nucleic acid sequences are substantially complementary is that the two sequences hybridize to each other under moderately stringent, or preferably stringent, conditions. Hybridization to filter-bound sequences under moderately stringent conditions may, for example, be performed in 0.5 M NaHPO₄, 7% sodium dodecyl sulfate (SDS), 1 mM EDTA at 65° C., and washing in 0.2×SSC/0.1% SDS at 42° C. (see Ausubel, et al. (eds), 1989, Current Protocols in Molecular Biology, Vol. 1, Green Publishing Associates, Inc., and John Wiley & Sons, Inc., New York, at p. 2.10.3). Alternatively, hybridization to filter-bound sequences under stringent conditions may, for example, be performed in 0.5 M NaHPO₄, 7% SDS, 1 mM EDTA at 65° C., and washing in 0.1×SSC/0.1% SDS at 68° C. (see Ausubel, et al. (eds), 1989, supra). Hybridization conditions may be modified in accordance with known methods depending on the sequence of interest (see Tijssen, 1993, Laboratory Techniques in Biochemistry and Molecular Biology—Hybridization with Nucleic Acid Probes, Part I, Chapter 2 “Overview of principles of hybridization and the strategy of nucleic acid probe assays”, Elsevier, N.Y.). Generally, stringent conditions are selected to be about 5° C. lower than the thermal melting point for the specific sequence at a defined ionic strength and pH.

In an embodiment, the toxin is a TMT substrate. As used herein, a “TMT substrate” can include several organic and inorganic compounds that can be methylated by TMT. The terms “TMT substrate” and “toxin” are used interchangeably herein. TMT substrates (or toxin) include, but are not limited to, compounds with the general formula I:
R—X⁻ I
wherein R is absent or is selected from the group consisting of H, inorganic side chains, organic side chains; and
wherein X⁻ is selected from the group consisting of S⁻ and a halide other than Fl⁻ (e.g. I⁻, Br⁻ and Cl⁻).

Examples of such TMT substrates are compounds containing sulfur (thiocyanates; e.g. thiocyanate ion), sulfides (e.g. ammonium sulfide and sulfide ion), bisulfide (e.g. bisulfide ion) and thiols or thiolates (e.g. thiosalicylic acid, thiobenzoic acid, 4,4′thiobisbenzenethiol, thiophenol) and halide-containing compounds (e.g. iodide ion, bromide ion, chloride ion, iodoaniline, iodobenzoic acid).

In another embodiment, the first cell of the method described above is tolerant to said toxin. “Tolerance” or “tolerant” as used herein refers to the capacity of a first cell to be less affected by a toxin than a second cell that does not have TMT activity or a TMT gene. Tolerant cells grow and develop better in the presence of a toxin when compared to intolerant cells, i.e. have increased growth and viability relative to intolerant cells.

The method also describes ranges of LD₅₀ of the toxin to be used. “Lethal dose 50” or “LD₅₀”, as used herein, refers to the amount of a toxin that stops or retards the growth of at least 50% of cells that do not express a recombinant TMT gene. Because of genetic variations among different types of organisms (and therefore, difference in intrinsic capacity to detoxify a particular toxin), ranges of LD₅₀ can vary significantly from one genus (or species) to another with respect to a specific toxin.

LD₅₀ for a given toxin with respect to a given cell or organism may be determined using standard methods known in the art. For example, LD₅₀ may be determined by contacting or culturing the cell or organism with increasing concentrations of the toxin, and in turn monitoring the growth of the cell or organism. The levels of growth observed in the presence of the respective concentrations of toxin may be used to determine the LD₅₀.

Similarly, a suitable toxin concentration or range thereof for use in the selection system described herein may be determined by assessing the growth of (1) a cell or organism comprising a nucleic acid encoding a polypeptide having TMT activity and (2) a corresponding cell or organism lacking said TMT activity; in the presence of increasing concentrations of the toxin. The concentration or range thereof resulting in a growth differential between (1) and (2) noted above would be suitable for use in the selection system described herein.

In an embodiment, the cell is a prokaryotic cell, and further, a bacterial cell (e.g. Escherichia coli). In a further embodiment, the LD₅₀ of said toxin is from about 150 to about 250 mM, in a further embodiment, from about 150 to about 200 mM. In another embodiment, the toxin concentration that is used for selecting a cell is from about 200 to about 250 mM, in a further embodiment, about 200 mM, and yet a further embodiment, about 250 mM.

In another embodiment, the cell is a plant cell. In a further embodiment, the toxin is contacted with the cell at a concentration from about 100 μM to about 10 mM, in further embodiments, at a toxin concentration selected from the group consisting of about 100 μM, 125 μM, 1 mM, 2 mM, 2.5 mM, 4 mM, 5 mM, 7.5 mM and 10 mM. In yet another embodiment, the toxin is contacted with said population of cells at a toxin concentration of more than about 5 mM, 10 mM or 20 mM.

In yet another embodiment, the cell is a eukaryotic cell, and further, an animal cell (e.g. mammalian cell, human cell, immortalized cell). In a further embodiment, the toxin is contacted with said population of cells at a toxin concentration from about 100 to about 150 mM, in a further embodiment, the LD₅₀ of said toxin is about 100 mM. In another embodiment, the toxin is contacted with said population of cells at a toxin concentration of greater than about 100 mM, 150 mM, or 200 mM.

It is understood that any value relating to a particular parameter (e.g. concentration) encompasses any variation, deviation or error (e.g. determined via statistical analysis) associated with a device or method used to measure the parameter. For example, in the case where the value of a parameter is based on a device or method which is capable of measuring the parameter with an error of ±10%, the value would encompass the range from less than 10% of the value to more than 10% of the value.

In a further embodiment, the present invention also provides a selectable marker system for the selection of a cell. As used herein, a “selectable marker” refers to a specific phenotypic trait conferred by the TMT gene that allows the selection of a first cell having the TMT gene from a population of cells. In an embodiment, the specific phenotypic trait is tolerance to a toxin (i.e. a TMT substrate).

Various genes and nucleic acid sequences of the invention may be recombinant sequences. The term “recombinant” means that something has been recombined, so that when made in reference to a nucleic acid construct the term refers to a molecule that is comprised of nucleic acid sequences that are joined together or produced by means of molecular biological techniques. The term “recombinant” when made in reference to a protein or a polypeptide refers to a protein or polypeptide molecule, which is expressed using a recombinant nucleic acid construct created by means of molecular biological techniques. The term “recombinant” when made in reference to genetic composition refers to a gamete or progeny or cell or genome with new combinations of alleles that did not occur in the parental genomes. Recombinant nucleic acid constructs may include a nucleotide sequence which is ligated to, or is manipulated to become ligated to, a nucleic acid sequence to which it is not ligated in nature, or to which it is ligated at a different location in nature. Referring to a nucleic acid construct as “recombinant” therefore indicates that the nucleic acid molecule has been manipulated using genetic engineering, i.e. by human intervention. Recombinant nucleic acid constructs may for example be introduced into a host cell by transformation. Such recombinant nucleic acid constructs may include sequences derived from the same host cell species or from different host cell species, which have been isolated and reintroduced into cells of the host species. Recombinant nucleic acid construct sequences may become integrated into a host cell genome, either as a result of the original transformation of the host cells, or as the result of subsequent recombination and/or repair events.

In a further embodiment, the invention also provides use of the nucleic acid encoding a TMT (e.g. a TMT gene) and the toxin for the production of a transgenic cell and/or transgenic organism. The production of a transgenic organism usually involves different steps depending on the characteristics of the organism to be genetically modified. In a first embodiment, the nucleic acid of interest, or “transgene”, is introduced into a cell. In an embodiment, the cell is a prokaryotic cell, and further, a bacterial cell. In another embodiment, the cell is an eukaryotic cell, and further a mammalian or plant cell (e.g. Medicago, Arabidopsis, tobacco, potato, rice, wheat, corn, canola, mustard, tomato, green pepper and bean cell, etc.). The transgene itself is usually introduced in combination with a selectable marker in the cell. In a further embodiment, the cell is selected for the presence of the transgene, based on the presence of the selectable trait (TMT gene). In order to produce progeny, the cell is then cultured in vitro (e.g. in a solid, semi-solid or liquid medium) or grown in vivo. Optionally, a selective pressure can be applied during the production of progeny or on the progeny itself to ensure the presence of the transgene of interest. In another embodiment, the selective pressure is applied on a plant or part of a plant comprising a transgenic cell in order to select a plant or part of a plant for the presence of the transgene of interest.

In a further embodiment, the cell is a callus cell. The nucleic acid of interest, in combination with the TMT gene, is first introduced into the callus cell either by bombarding the cell with the transgene of interest or by infecting the cell with a bacterium (e.g. Agrobacterium). In an embodiment, the transgene of interest and the TMT gene have been inserted into Agrobacterium. As such, upon infection of the callus cell, the transgene of interest and the TMT gene will transfer from Agrobacterium to the callus cell (e.g. cytoplasm and/or nucleus). The transgenic callus cell can then be selected for the presence of the TMT gene (and the presence of the nucleic acid of interest). The selected transgenic callus cell can further be grown into a plant when transferred onto an appropriate medium

In another embodiment, the transgene is introduced into a cell within an organism. When the cell is a plant cell, the transgene and the selectable marker can be introduced in parts of the plants (e.g. roots). Optionally, this can be achieved using bombardment, viral- or bacterial-induced transformation. Transgenic cells having the transgene of interest and the selectable marker will thus be more tolerant to the toxin than non-transgenic cells. Consequently organisms having a transgenic cell will be more tolerant than non-transgenic organisms to the toxin, thereby providing a selection criterion.

In a further embodiment, the selectable marker can be designed to autonomously replicate within a host cell and/or it can be engineered to become integrated into the genome of the host cell.

The invention further relates to a plant cell comprising a nucleic acid comprising a recombinant vector comprising a first nucleic acid which encodes a polypeptide having TMT activity. The invention further relates to a transgenic plant, or part thereof, or progeny or seed thereof, comprising said recombinant vector.

Although various embodiments of the invention are disclosed herein, many adaptations and modifications may be made within the scope of the invention in accordance with the common general knowledge of those skilled in this art. Such modifications include the substitution of known equivalents for any aspect of the invention in order to achieve the same result in substantially the same way. Numeric ranges are inclusive of the numbers defining the range. In the claims, the word “comprising” is used as an open-ended term, substantially equivalent to the phrase “including, but not limited to”. The following examples are illustrative of various aspects of the invention, and do not limit the broad aspects of the invention as disclosed herein.

### EXAMPLES

### Example 1

### Expression of TMT in E. coli

The full-length copy of TMT1 was generated by PCR using specific primers corresponding to amino acid residues 1-9 (Primer I: 5′ GAGAGGATCCGTGGCTGAGGAACAACAAAAAGCAGG 3′ (SEQ ID NO: 7)) and 223-230 (Primer II: 5′ GAGAGGTACCTCAATTGATCTTCTTCCACCTCCC 3′ (SEQ ID NO: 8)) of the TMT1 gene. A point mutation was introduced in Primer I, substituting the A in the initial start codon of the TMT gene to a G (i.e. a variant of the TMT1 cDNA of SEQ ID NO: 1 having a G replacing the A of the ATG start codon). GAGA sequences were added as protector sequences and restriction endonuclease cleavage sites (underlined) for BamHI and KpnI were introduced at the 5′ends of both primers to facilitate eventual cloning. PCR was carried out using TMT1 cDNA (obtained from screening a cDNA library of Brassica oleracea leaves with an Arabidopsis thaliana EST [AC 3212850]; Attieh et al. [2002], Plant Mol. Biol. 50:511-521) as follows: activation for 15 min at 95° C., 30 cycles of denaturation at 94° C. for 1 min, annealing at 58° C. for 1 min, extension at 72° C. for 1 min and a final extension at 72° C. for 10 min was also included. The amplified fragment was subjected to electrophoresis, purified with the Qiaquick™ Gel Extraction Kit (Qiagen) and digested with BamHI and KpnI. The purified and digested fragment was then cloned into the pQE30 expression vector containing a 6-His tag. The plasmid containing the TMT insert was called pQETMT1. Plasmids pQE30 (vector only, no insert) and the PQETMT1 (TMT1 insert) were transformed into M15 (pREP4) Escherichia coli cells.

E. coli M15 cells transformed with the empty vector pQE30 (referred to as non-transgenic bacteria) or pQETMT1 (hereafter referred to as transgenic bacteria) were grown as 15 ml starter cultures in LB medium supplemented with 100 μg/ml ampicillin and 25 mg/ml kanamycin at 37° C. for 16 h. 5 ml of each culture was then transferred to 100 ml of fresh LB medium containing the same concentrations of both antibiotics and cultures grown to an OD₆₀₀ of 0.6. An appropriate volume of each culture was transferred to culture tubes containing different concentrations of either KI or NaSCN (ranging from 0 to 250 mM and 0 to 200 mM respectively) in a final volume of 5 ml. In the culture tubes, expression of the recombinant TMT protein was induced by adding β-D-thiogalactopyranoside (IPTG) to a final concentration of 1 mM. All the culture tubes were incubated at 37° C. for another 4 h. Bacterial growth at different toxin concentrations was measured by optical density (OD₆₀₀) and the difference in OD₆₀₀ (ΔOD₆₀₀) was calculated.

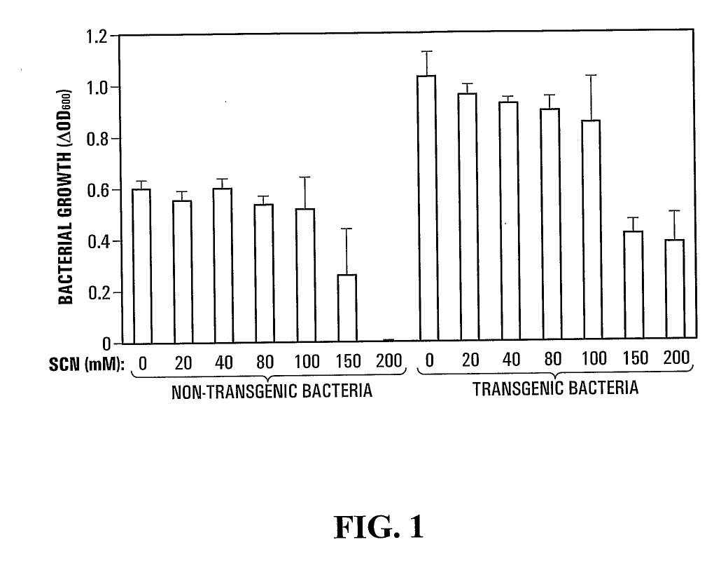

As shown in FIG. 1, transgenic bacteria grew faster than the non-transgenic bacteria in the presence of NaSCN. At a concentration of 200 mM NaSCN, the presence of the toxin halted non-transgenic bacterial growth whereas the transgenic bacteria continued to grow (ΔOD₆₀₀ of 0.377).

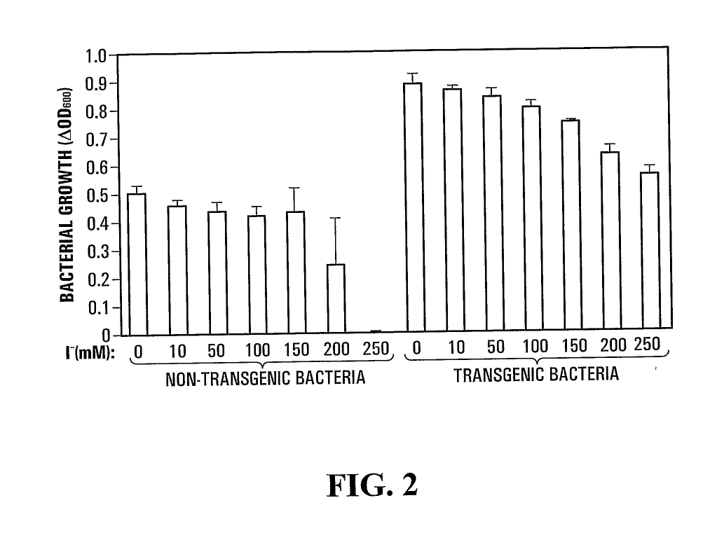

A similar phenomenon was observed for transgenic and non-transgenic bacteria grown in the presence of KI (FIG. 2). In the presence of a range of concentrations of KI, transgenic cells expressing TMT grew faster than non-transgenic cells. At 250 mM KI, while the growth of the non-transgenic bacteria was halted, the transgenic bacteria continued to grow (ΔOD₆₀₀ of 0.547).

### Example 2

### Expression of TMT in Potato Roots

pBluescript plasmid harboring the full-length TMT1clone (SEQ ID NO:1) was linearized with BamHI and KpnI and cloned in a sense orientation into the binary pBin19 double enhancer vector between the 35S promoter of CaMV and the Nos terminator. Agrobacterium rhizogenes wild-type strain A4 was then transformed either with the pBin/TMT vector or pBin vector alone. Freshly cut Solanum tuberosum cv Russet Burbank petioles were decontaminated by soaking in diluted commercial bleach (10%) containing 0.01% Tween-20™ for 10 min, followed by three 10 min washes in sterile water. The petioles were then cut into 4 cm segments and cultured apical side down in MS medium containing 0.4% phytagel™. The upper parts of the petioles were infected with freshly streaked A. rhizogenes A4 carrying the pBin19 vector alone (non-transgenic roots) or with the pBin vector with the TMT insert (transgenic roots). Roots started appearing after about 10 days. Each root was considered as a separate clone. They were initially transferred in small Petri dishes containing MS/0.2% phytagel and carbenicillin (50 mg/ml). Carbenicillin is bacteriostactic and therefore progressively frees the medium of A. rhizogenes.

Every 2 weeks, the carbenicillin concentration was halved until no carbenicillin was present in the medium. All roots were then screened for TMT activity using an in vivo technique where KI in solution (100 mM) was added to the roots and emission of CH₃I was detected using gas chromatography. Several clones with varying TMT activity were selected for growth analysis on MS supplemented with different concentrations of KI and NaSCN (ranging from 0 to 200 mM). 5 mm of meristem from 1 week-old sub-cultured roots was used for the growth assay. The roots were placed in MS containing 0.2% phytagel and allowed to grow for 10 days after which the growth was measured using WinRhizo™ software.

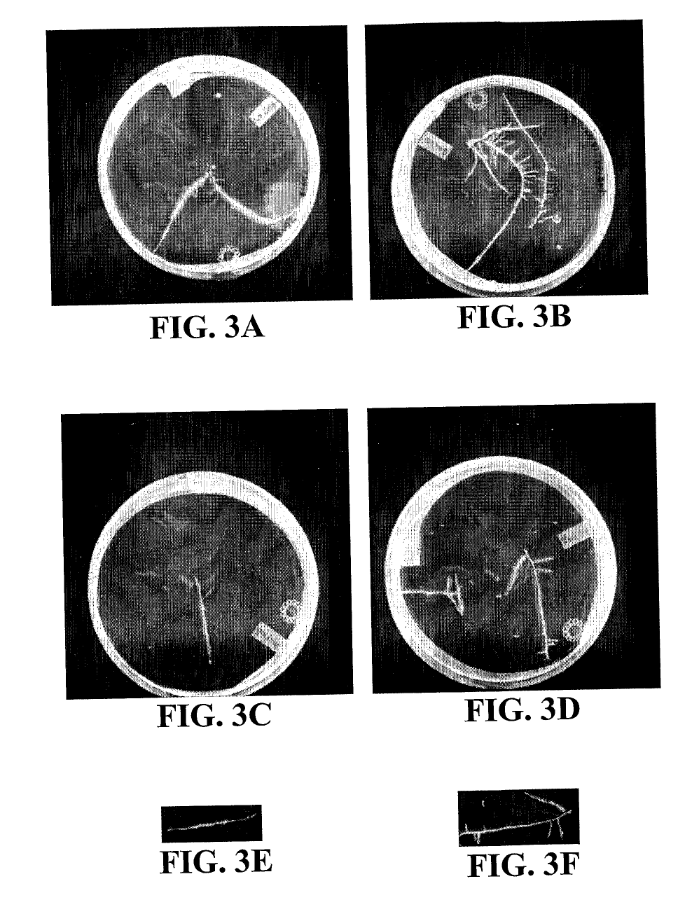

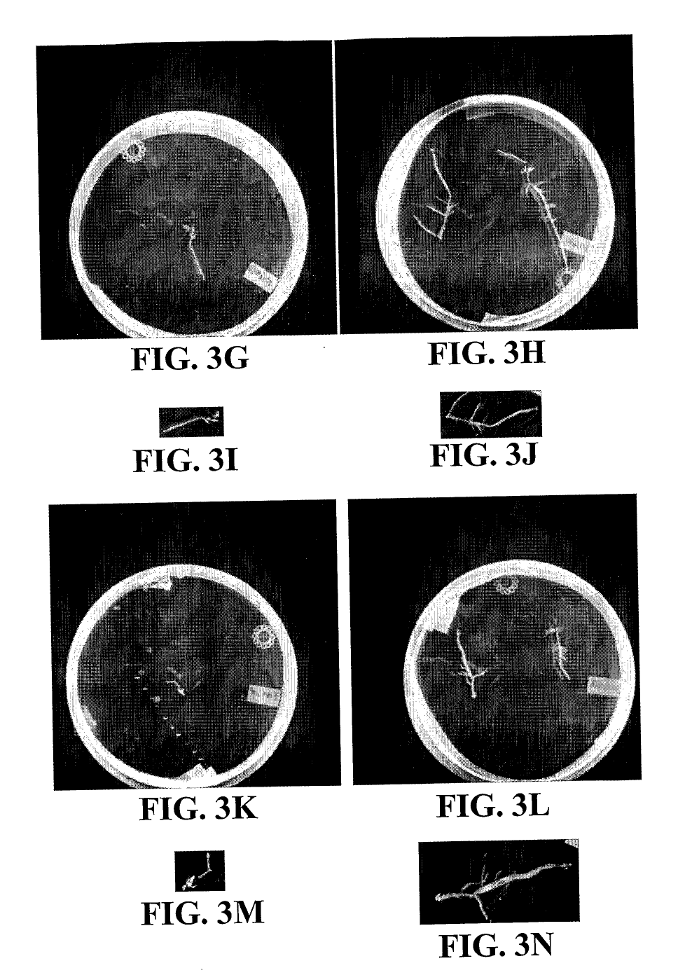

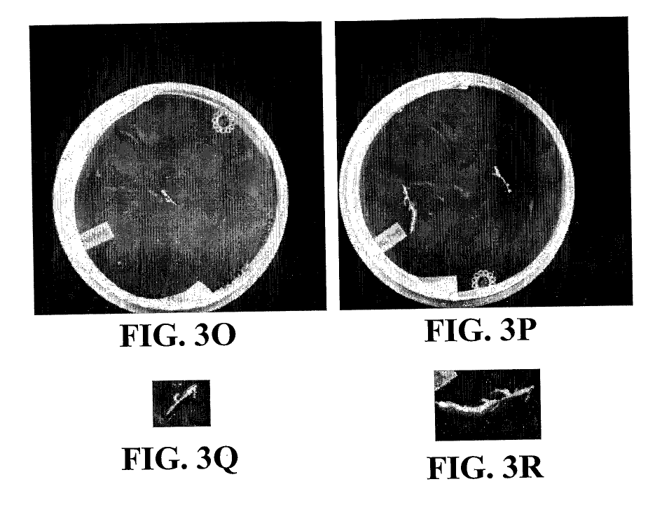

Longer transgenic roots were measured in all SCN concentrations tested (FIG. 3). As the toxin concentration increased, the growth of both transgenic and non-transgenic roots was affected, however, transgenic roots were longer than non-transgenic roots.

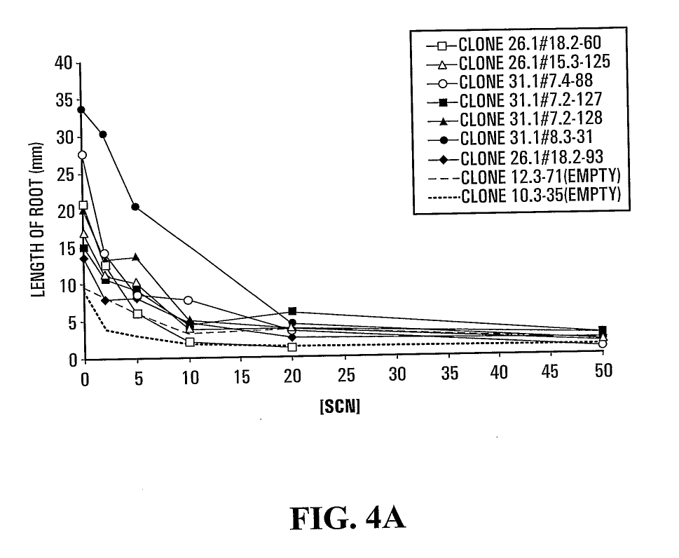

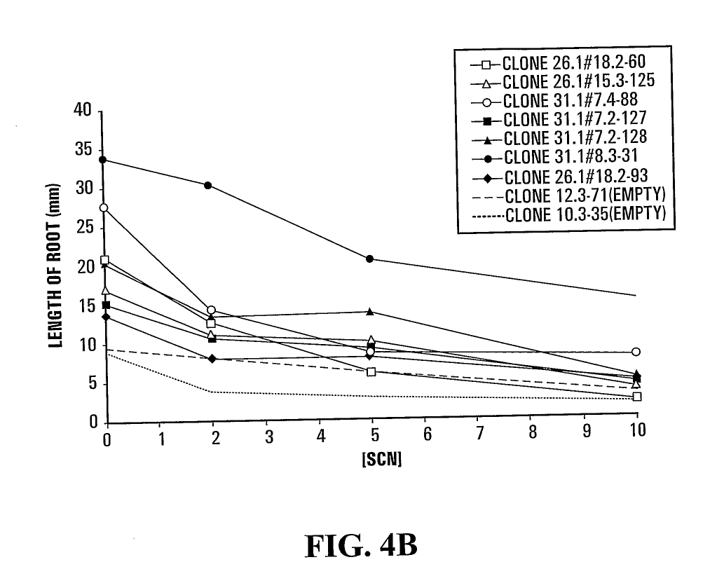

This phenomenon can also be observed in FIGS. 4A and 4B. For all toxin concentrations, non-transgenic roots (represented by broken lines) were shorter that transgenic roots (represented by solid lines).

### Example 3—Expression of TMT in a Eukaryotic Cell Line

A TMT insert was generated by linearizing pBluescript vector harboring the full-length clone (described in Example 2) with SpeI and ApaI and inserted into the pTracer™-SV40 mammalian expression plasmid. This vector contains a cycle-3-GFP, an improved Green Fluorescent Protein gene fused to Zeocin™ resistance gene for selection in mammalian cells. Transfection of HEK 293 cells was carried out in 24 well plates using Fugene 6™ Transfection reagent (Roche Molecular Biochemicals). A ratio of DNA to Fugene of 1:6 and 200 or 400 ng of DNA were used. Each well was seeded with 2×10⁵ cells grown in a monolayer for 16 h in 500 l DMEM containing 10% serum. Transfection was performed on 90% confluent cells. Prior to transfection, the medium containing serum was removed and replaced with 150 μl of serum-free medium in each well. A DNA/Fugene 6 mixture was incubated for 15 min at room temperature and 100 μl of this mixture was added drop-wise to each well while cells were gently rocked. After a 3 hour incubation, another 250 μl DMEM containing 2× serum was added to each well and cells were further incubated for 48 h. Medium was than replaced with fresh medium containing different concentrations of KI or NaSCN (ranging from 0-200 mM). These cells were allowed to grow for another 24 h. Growth was monitored by fluorescent microscopy to detect the presence of GFP. Medium was than removed from all wells and cells were lysed with the buffer provided in the Fugene 6 reagent kit to provide a total protein extract. An aliquot of this extract was used in an in vitro assay to monitor the emission of CH₃I. Briefly the aliquots were incubated with 20 mM KI diluted in buffer A (100 mM Tris-acetate pH 7.5, 10% glycerol and 0.01% β-mercaptoethanol) and 0.5 mM S-adenosyl-L-methionine (SAM). The emission of CH₃I was then monitored by gas chromatography.

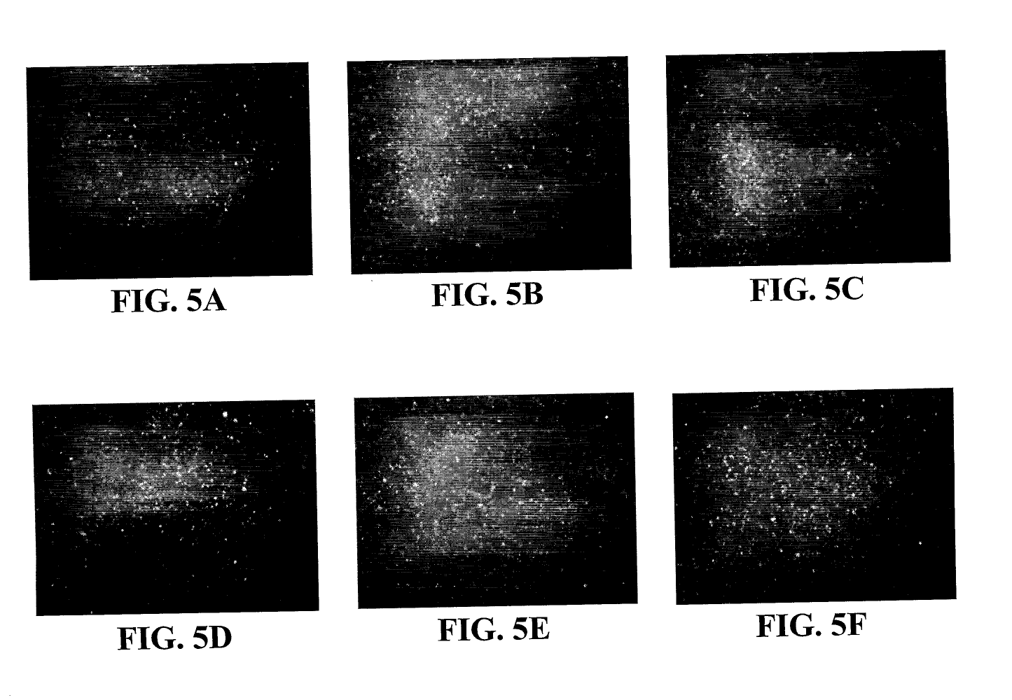

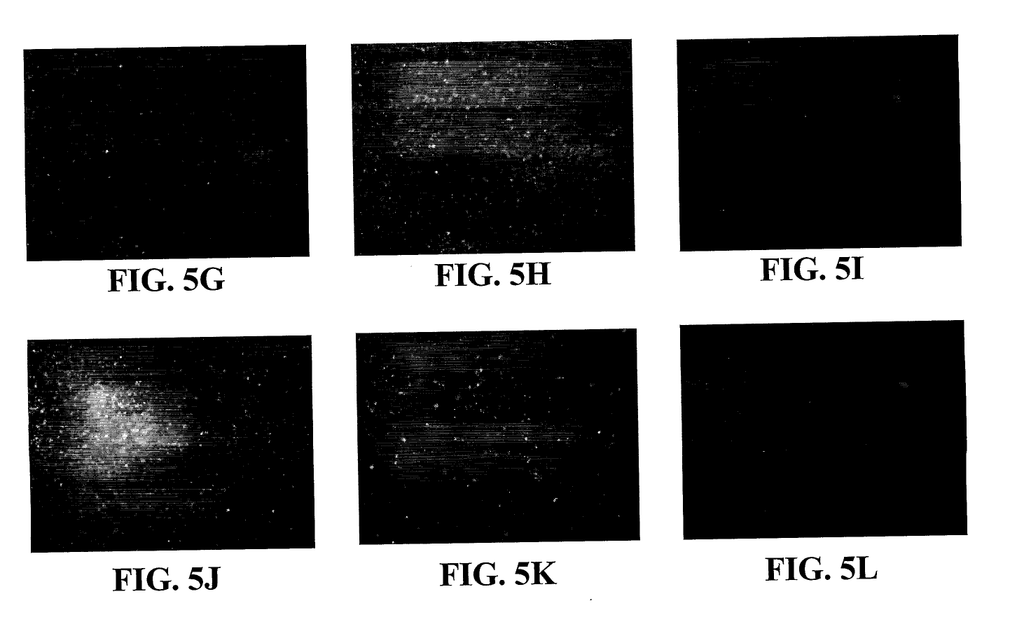

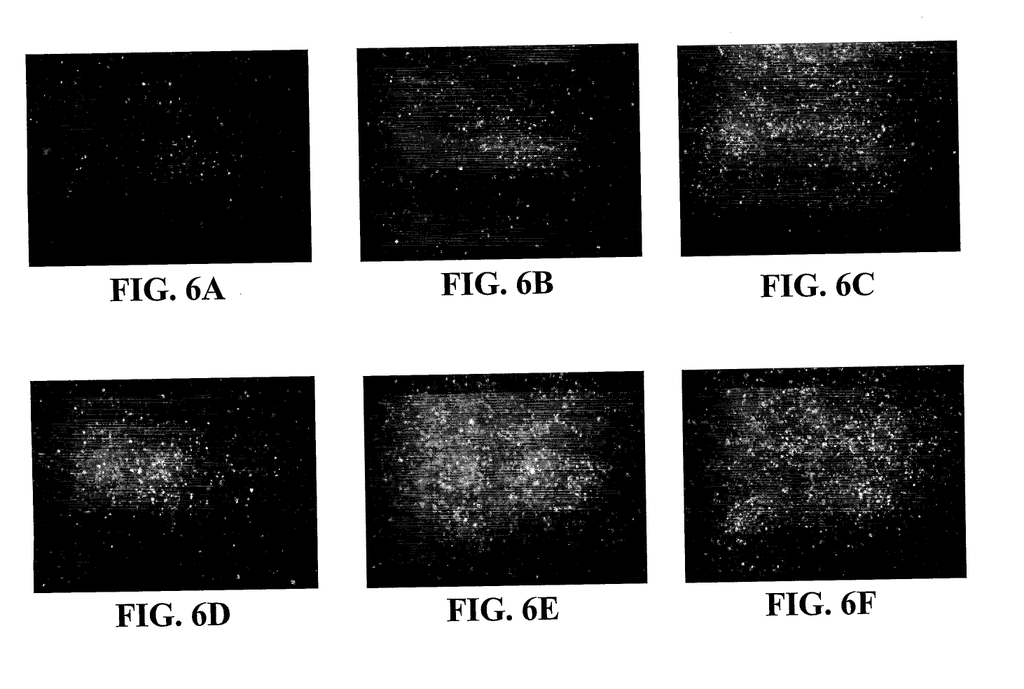

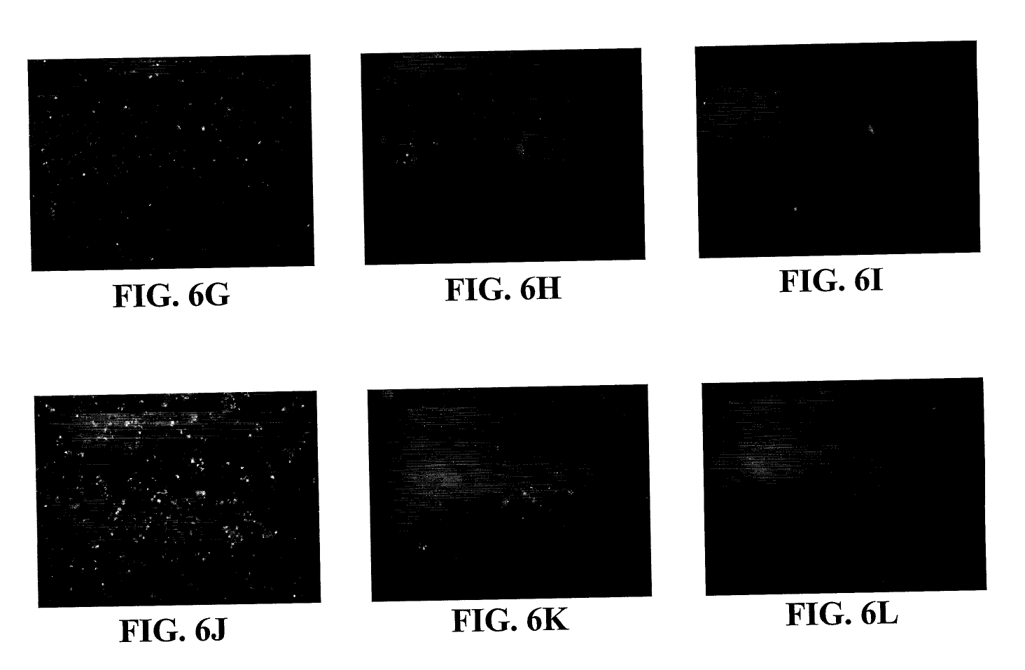

As shown in FIGS. 5 and 6, there was more fluorescence in the transgenic than in the non-transgenic cells at any given concentration of either NaSCN or KI. These results suggest that, in the presence of the toxin, cell viability was increased in transgenic cells when compared to non-transgenic cells.

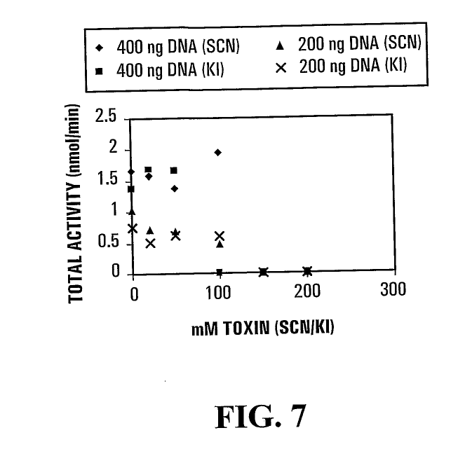

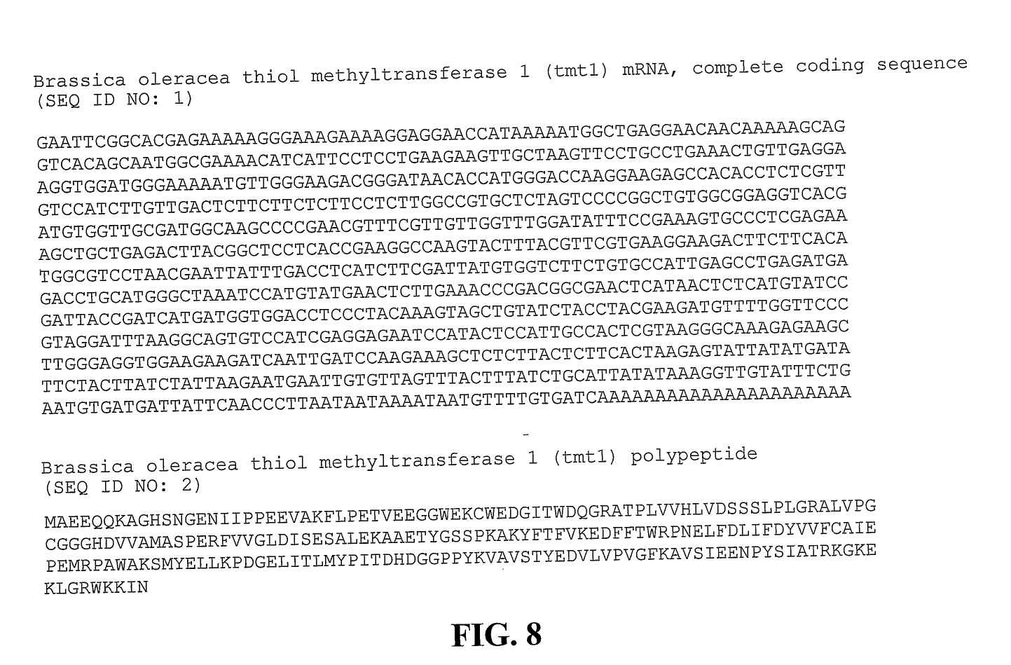

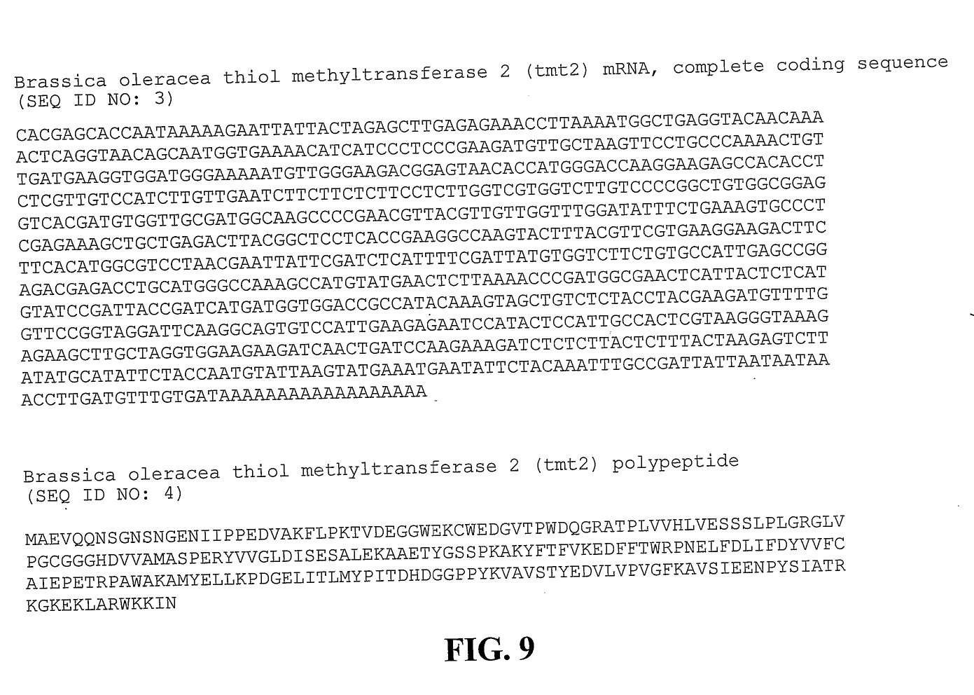

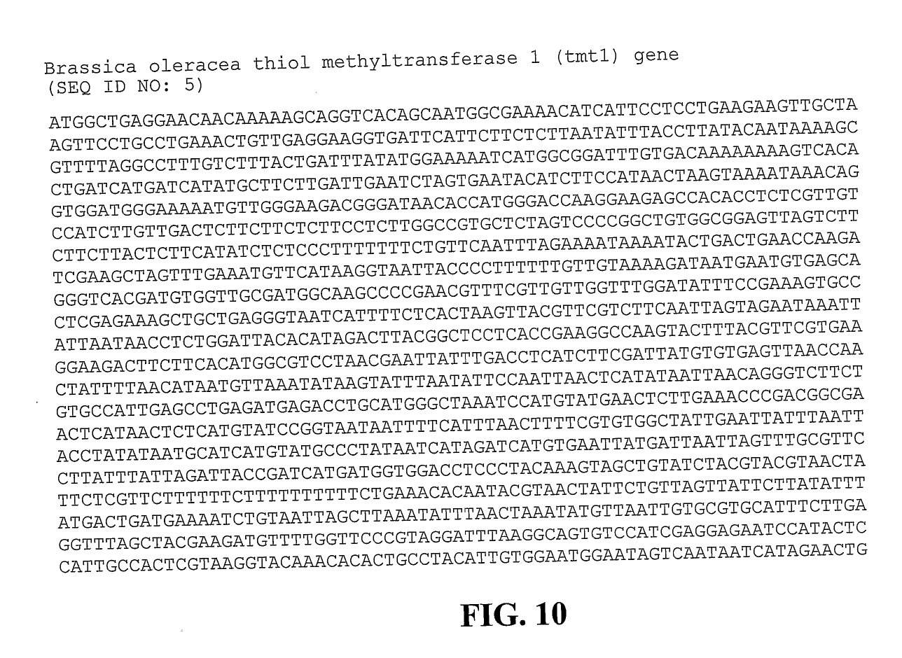

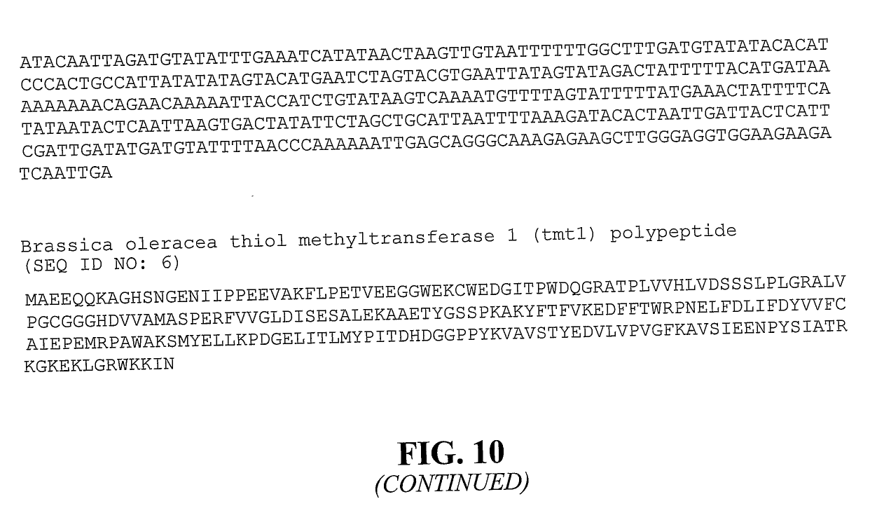

The results presented in FIG. 7 suggest that a higher TMT activity was measured in the cells that had been transfected with 400 ng of DNA as compared to those transfected with 200 ng DNA.

### Example 4—Assaying Methyltransferase Activity

In vitro assay: I⁻ methylation is used to assay TMT activity. For this in vitro (extractable) activity assay, a standard assay mixture contains 100 mM Tris-acetate buffer, pH 7.3, 14 mM β-mercaptoethanol, 10% glycerol (v:v), 0.5 mM AdoMet, 50 mM KI, and up to 1 mg protein in a total assay volume of 0.5 ml. The mixture is incubated for 30 min at room temperature, and the product, CH₃I, is quantified by analyzing headspace samples by gas chromatography as described in Attieh et al. ([1995] Journal of Biological Chemistry, 270:9250-9257). In the assay for HS⁻ methylation, 20 mM (NH₄)₂S replaces KI in the above assay.

Radiometric assay: This alternative in vitro activity assay, adapted from thiol methyltransferase assay of Borchardt and Cheng ([1978] Biochimica and Biophysica Acta, 522:340-353), is based on the ability of the purified enzyme to transfer the ³H—CH₃ group of [methyl-3H]AdoMet to thiol compounds. The standard assay mixture contains 75 mM Tris-acetate buffer, pH 7.3, 14 mM β-mercaptoethanol, 10% glycerol (v:v), 25 μM AdoMet (SO₄²⁻ salt), 0.01 μCi [methyl-³H]AdoMet (SO₄²⁻ salt) and 200 μM methyl acceptor (thiol or thiolate substrates) in a total volume of 100 μl. The reaction is started by adding the enzyme purified to homogeneity (Attieh et al., 1995). After 30 min at room temperature, the reaction is stopped by adding 50 μl of 10 M NaOH. The mixture is transferred to 16×125 mm glass culture tubes, and methylated products are extracted with 5 ml toluene, or with 5 ml hexane when thiocyanate is the substrate. In assays containing organic acids as the substrates, the mixture is acidified with 50 μl of 10 M HCl prior to extraction. After centrifugation for 5 min at 3000 g, a 3 ml aliquot of the organic phase is mixed with an equal volume of scintillation cocktail and counted for radioactivity. Assay mixtures with boiled enzyme or without the methyl acceptor are used as blanks, and the data are corrected accordingly.

In vivo assay: This assay was used in the studies described herein. In vivo TMT activity is measured in 10 leaf disks placed on a Whatman filter paper (4.25 cm) in a 50-ml Erlenmeyer flask, and incubated with 2 ml of a 100 mM KI solution (Saini et al., [1995] Plant, Cell and Environment, 18:1027-1033). The flask is sealed with a rubber stopper and CH₃I formed in the headspace is quantified after 2 h by gas chromatography. Assays for alternative substrates or inhibitors of the enzyme contained 20 mol m⁻³ KI (the known substrate) and a range of concentrations of the compound to be tested. In the assay for HS⁻ methylation, KI in the above assay is replaced with 20 mM (NH₄)₂S.

### Example 5

### Use of TMT as a Selectable Marker in Dicot Root Systems

Methodology: pBluescript plasmid harboring the full-length TMT-1 clone (see above) was linearized with BamHI and KpnI and cloned in a sense orientation into the binary PBin19 double enhancer vector between the 35S promoter of CaMV and the Nos terminator. The PBin/TMT vector and PBin alone were then transformed into Agrobacterium rhizogenes wild-type strain A4. Freshly cut petioles were decontaminated by soaking in diluted commercial bleach (10% v/v) containing 0.01% (v/v) Tween-20 for 10 min, followed by 3×10 minutes washes in sterile water. The petioles were then cut into 4 cm segments and cultured apical side down in MS medium containing 0.4% (w/v) phytagel. The upper parts were infected with freshly streaked A. rhizogenes A4 carrying the PBin19 vector with or without the TMT insert. On average, roots started appearing after about 10 days, but in some species like nasturtium, roots appeared after only 5 days on infection, while in basil, roots only appeared after 2-3 weeks. Each root was considered as a separate clone. They were initially transferred in small Petri dishes containing MS/0.2% phytagel and carbenicillin (50 mg/ml). Carbenicillin is a bacteriostactic and therefore progressively frees the medium of A. rhizogenes. Every 2 weeks, the carbenicillin concentration was halved until no carbenicillin was present in the medium.

At this stage, about 8-9 root clones (mixture of fast and slow growing clones) were randomly chosen from the whole population of clones. They were marked A1, A2, etc. and therefore none of the operators knew whether these clones had been transformed with the empty vector or the vector/TMT gene construct. Each of the clones chosen was sub-cultured for 7 days after which young meristems (0.6 cm) were cut and transferred to big petri dishes (15 cm) containing different concentrations of SCN included in the MS/0.2% phytagel mix. Each petri dish was divided into two parts so that on each side of the dish, two meristems from the same clone could be compared. The root segment was placed in the centre of the plate with the meristem end facing outwards. Each plate therefore contained all 8-9 clones chosen (aligned next to each other with about 1 cm gap between clones) and on each plate, duplicates of the same clone were done. Each experiment was done by two different operators, giving a total of 4 replicates for each clone on each concentration of SCN chosen. After another 7 days of growth, the mean root length was measured using Winrhizo™ software. The mean root length is indicated in the appended figures for all species tested (see Table 1) From the Winrhizo™ results, the operators were asked to choose ‘their’ positive clones (see Table 1), shown as filled, black bars in the bar graphs in the appended Figures.

To verify whether the clones (still unknown to operators) selected based entirely on the difference in growth were really transformed, DNA was extracted using the DNeasy™ plant kit (Qiagen). Two sets of PCR were done: (1) firstly with primers Kan^{R}F (5′ GTCATTTCGAACCCCAGAGTC 3′ [SEQ ID NO: 9]) and Kan^{R}R (5′CTGAATGAACTGCAGGACGAG 3′ [SEQ ID NO: 10]) against the kanamycin resistance gene (since the binary PBin19 double enhancer vector contains kanamycin gene) and (2) with TMT specific primers (region corresponding to TMT shown in bold italics) ASCF (5′ GAGAGGTACCATGGCTGAGGAACAAAAAAAGCAGG 3′ [SEQ ID NO: 11]) and ASCR (5′ GAGAGGATCCTCAATTGATCTTCTTCCACCTCCC 3′ [SEQ ID NO: 12]). The former primers give a PCR product of around 700 bp, while the latter primers amplify the full length TMT also giving a PCR product of 681 bp. PCR results are indicated in the appended figures for all species tested (see Table 1).

To confirm that these selected clones possessed the active protein, all the clones were screened for TMT activity using an in vivo technique where 100 mM KI in solution (a TMT substrate) was added to the roots and emission of CH₃I was quantified using gas chromatography. TMT activity results are shown in the appended figures for all species tested (see Table 1), where the activity is given on the right Y-axis while the left Y-axis gives the Winrhizo™ data. In the appended figures, the original clone number (hidden all the time from the operators) is given.

TABLE 1
Reference to Figures containing results
of dicot root studies described herein

| | | Corresponding Figure(s) | |
|---|---|---|---|
| | Positive | | PCR results |
| | clones chosen | Root length & | with TMT- |
| Species | by operator | TMT activity | specific primers |
| Sunflower | 26.1#15.5 | | |
| | 26.1#15.25 | | |
| | 26.1#15.26 | | |
| Tobacco | 26.1#15.6 | | |
| | 31.1#7.8 | | |
| | 26.1#15.11 | | |
| | 31.1#7.74 | | |
| | 31.1# 7.5 | | |
| Carrot | 31.1# 7.28 | | |
| | 26.1#15.30 | | |
| | 26.1#15.57 | | |
| | 26.1#15.56 | | |
| nasturtium | 31.1#7.5 | | |
| | 26.1#15.3 | | |
| | 26.1#15.27 | | |
| | 26.1#15.5 | | |
| | 31.1#7.7 | | |
| Basil | 26.1#15.42 | | |
| | 31.1#7.76 | | |
| Potato | 26.1#18.2-60 | | |
| | 26.1#15.3-125 | | |
| | 31.1#7.2-127 | | |
| | 31.1#7.2-128 | | |
| | 31.1#8.3-31 | | |
| | 26.1#18.2-93 | | |
| Green bean | 31.1# 7.23 | | |
| | 31.1# 7.19 | | |
| | 31.1# 7.26 | | |
| Tomato | 31.1# 7.102 | | |
| | 31.1# 7.117 | | |

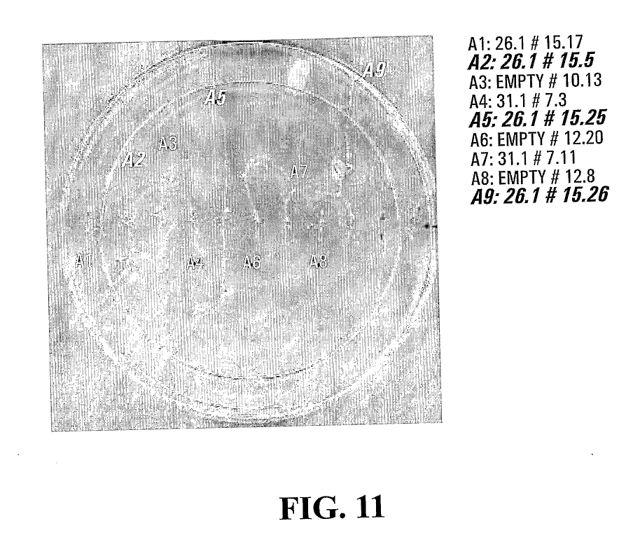

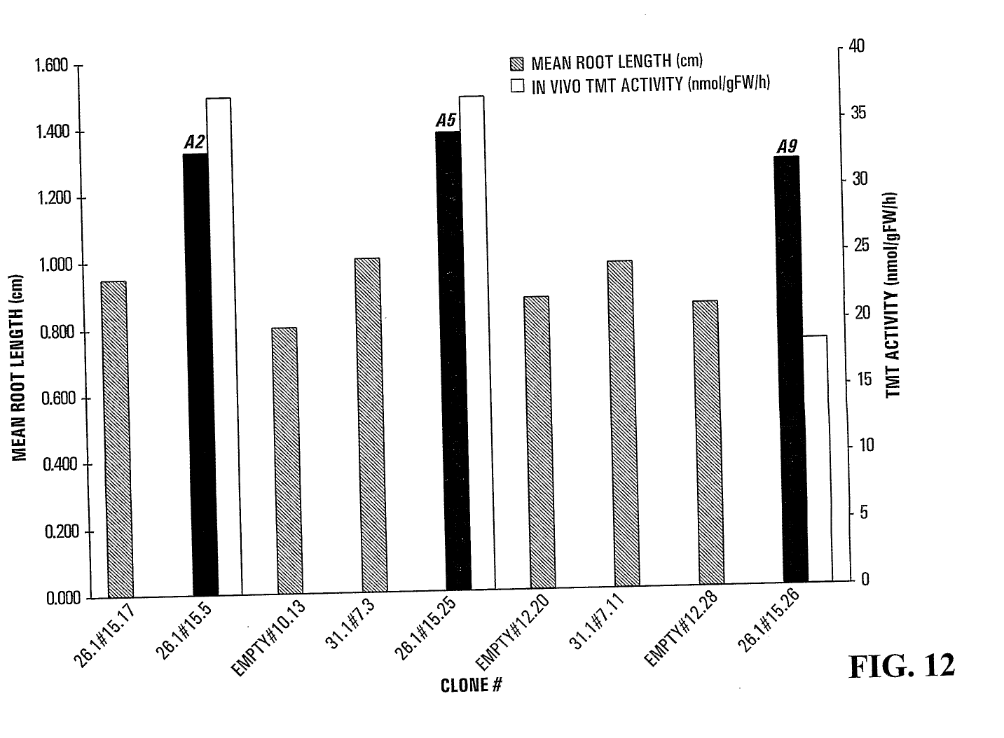

With reference to FIG. 11, the Petri dish shown contains 10 mM SCN and the roots are the individual sunflower root clones randomly chosen for assay. From these results, root clones A2, A5 and A9 were selected as putative positive based solely on their greater length. As shown in FIG. 12, all 3 clones chosen visually were determined to be positive for TMT activity.

With reference to FIG. 12, all 3 sunflower root clones chosen on the basis of length (solid-filled bars) were determined to be TMT-positive. Therefore, clones that were deemed positive chosen upon visual examination were all TMT-positive.

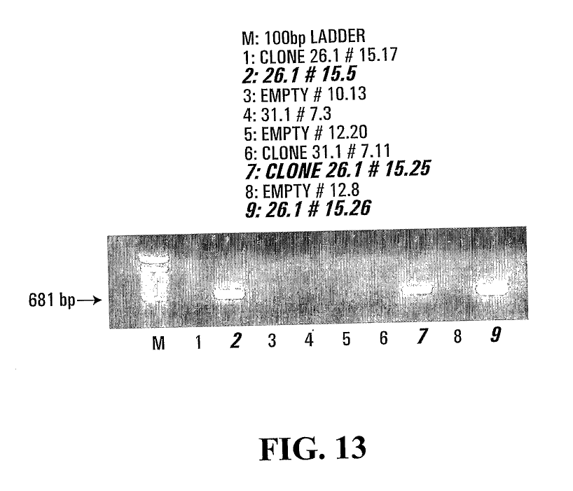

With reference to FIG. 13, PCR using TMT-specific primers showed that all 3 sunflower root clones chosen on the basis of length (see FIGS. 11 and 12) did possess the TMT gene proving that the iodide methylation into CH₃I was indeed due to the TMT gene introduced into those clones. This data correlates with the activity data shown FIG. 12. All the lanes where no product was obtained indeed did not show any TMT activity (see FIG. 12).

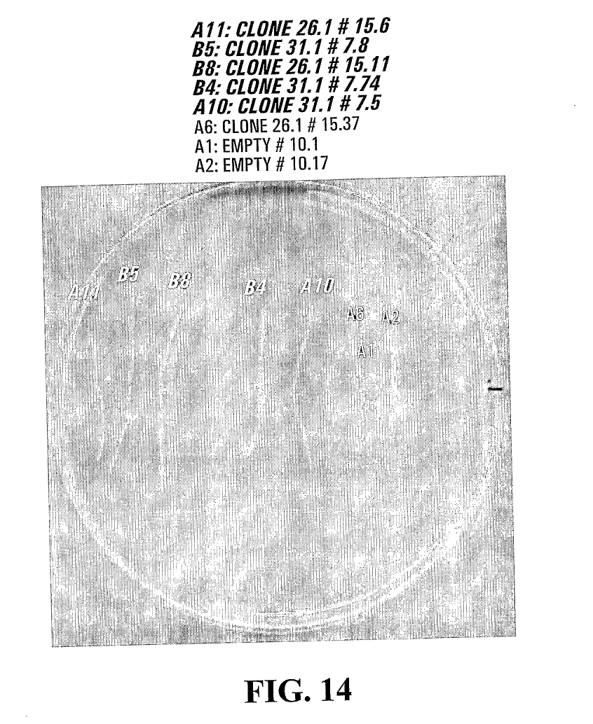

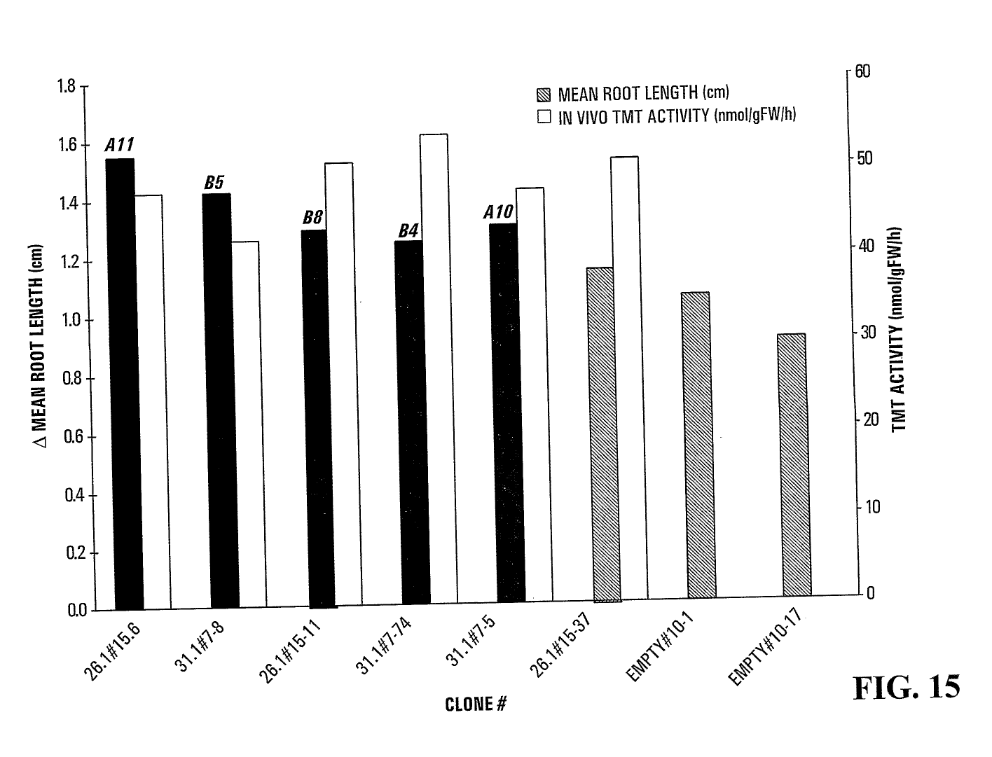

With reference to FIG. 14, the Petri dish shown contains 4 mM SCN and the roots are the individual tobacco root clones randomly chosen for assay. From this assay, root clones A11, B5, B8, B4 and A10 were selected as putative positive based solely on their greater length. All 5 clones chosen visually turned out to be positive for TMT activity (See FIG. 15).

With reference to FIG. 15, all 5 tobacco root clones chosen on the basis of length (solid-filled bars) turned out to be TMT-positive. Therefore, clones that were deemed positive upon visual examination were all TMT-positive. Clone 26.1 #15.37 which was not selected through visual screening, turned out to be positive for TMT indicating a 100% transformation efficiency in tobacco.

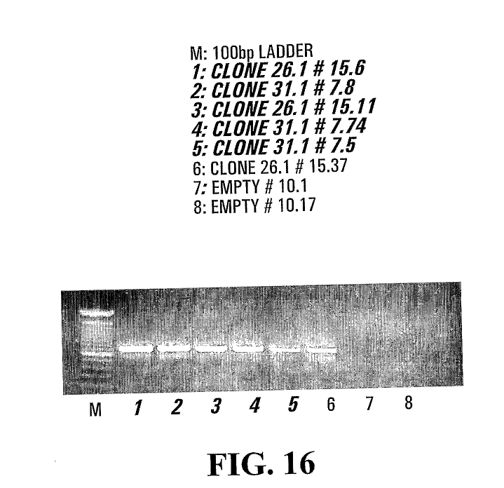

With reference to FIG. 16, PCR using TMT-specific primers showed that all 5 tobacco root clones chosen on the basis of length (see FIGS. 14 and 15) did possess the TMT gene proving that the iodide methylation into CH₃I was indeed due to the TMT gene introduced in those clones. Clone 26.1 #15.37 (lane 6), which was not selected through visual screening, turned out to be positive for the TMT gene as well consistent with the presence of TMT activity shown in FIG. 15. All empty clones (lanes 7-8) were negative, consistent with the data of FIGS. 14 and 15.

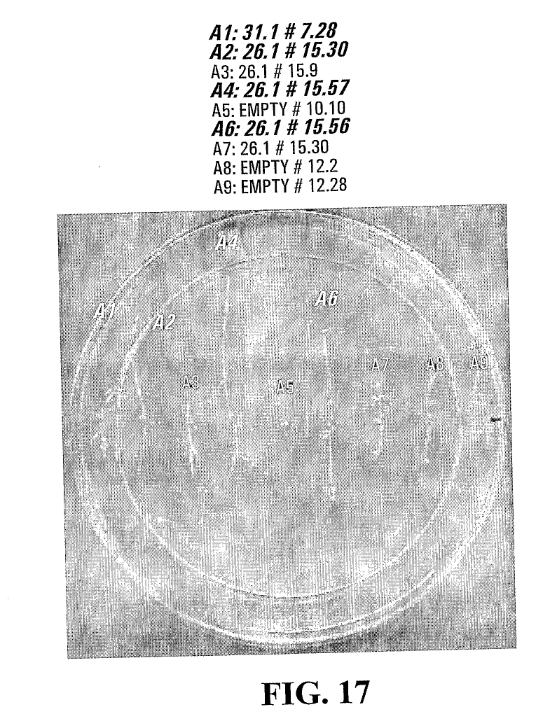

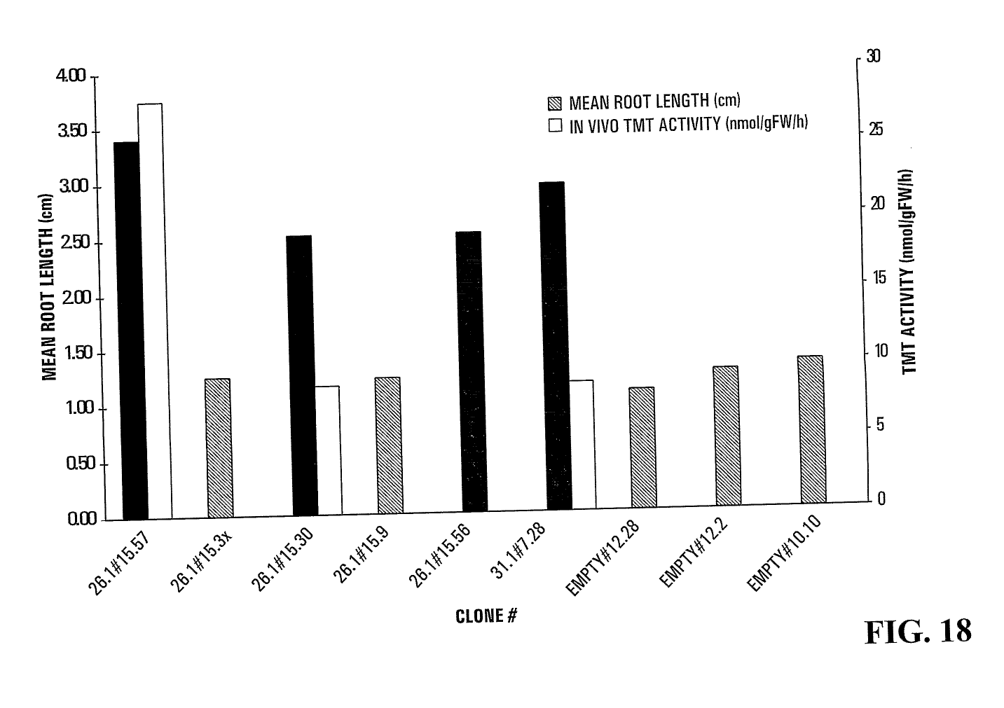

With reference to FIG. 17, the Petri dish shown contains 125 μM SCN and the roots are the individual carrot clones randomly chosen for assay. From this assay, root clones A1, A2, A4 and A6 were selected as putative positive based solely on their greater length. 3 of the 4 clones chosen visually were shown to be positive for TMT activity (see FIG. 18).

With reference to FIG. 18, 3 out of the 4 carrot root clones chosen on the basis of length (solid-filled bars) were found to be TMT-positive.

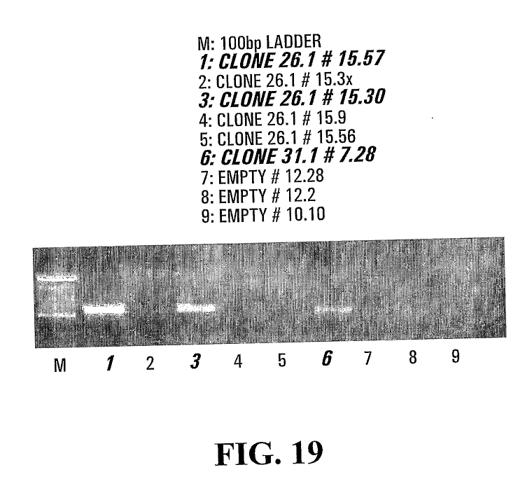

With reference to FIG. 19, PCR using TMT-specific primers confirmed the activity results, and out of the 4 carrot clones chosen on the basis of length, 3 did possess the TMT gene proving that the iodide methylation into CH₃I was indeed due to the TMT gene introduced in those clones.

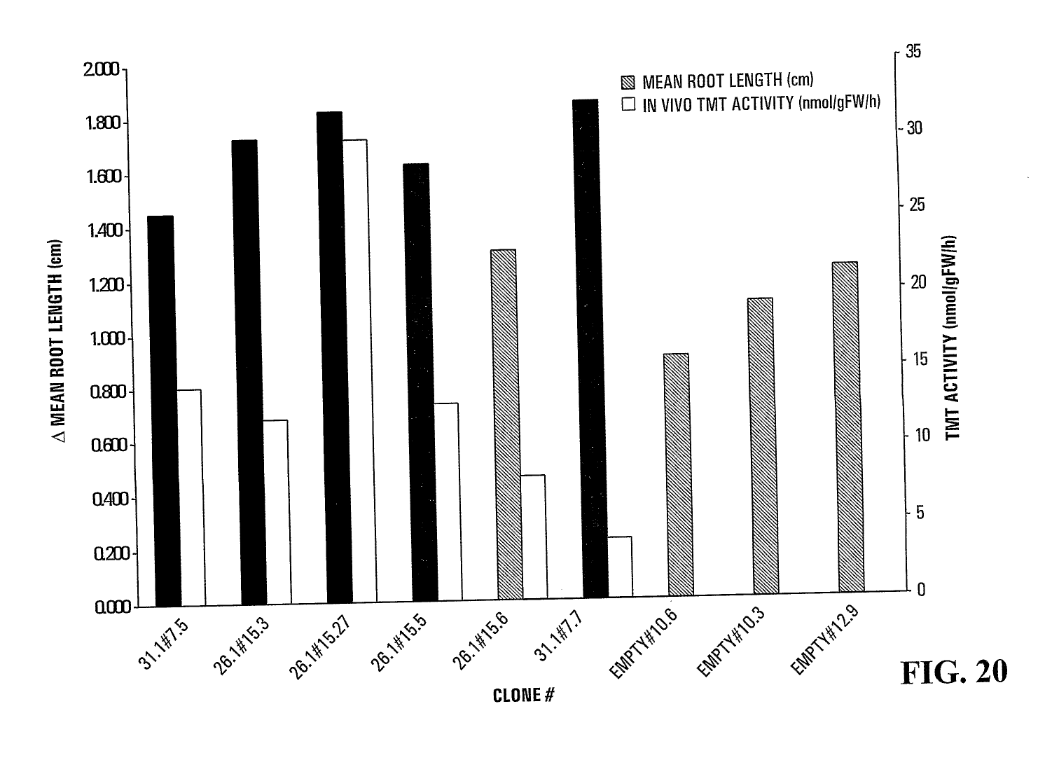

With reference to FIG. 20, all 5 nasturtium root clones chosen on the basis of length (solid-filled bars) turned out to be TMT-positive. Clone 26.1 #15.6, which was not selected through visual screening, turned out to be positive for TMT. Transformation efficiency in this species was therefore 100%. Therefore, clones that were deemed positive chosen upon visual examination were all TMT-positive.

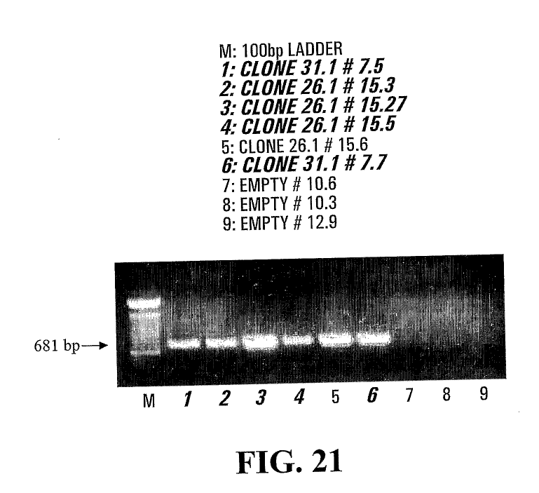

With reference to FIG. 21, PCR using TMT-specific primers showed that all 5 nasturtium root clones chosen on the basis of length (see FIG. 20) did possess the TMT gene proving that the iodide methylation into CH₃I was indeed due to the TMT gene introduced in those clones. Clone 26.1 #15.6 (lane 5), which was not selected through visual screening, turned out to be positive for the TMT gene as well explaining the TMT activity shown in Figure. All empty clones (lanes 7-9) were negative.

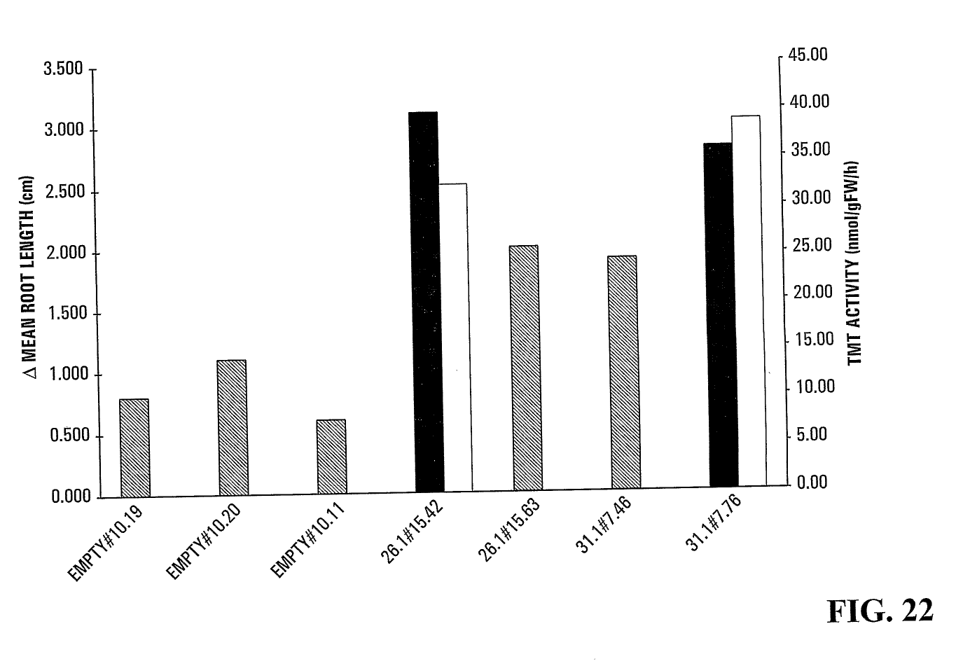

With reference to FIG. 22, both basil root clones chosen on the basis of length were determined to be TMT-positive. Clones 26.1 #15.63 and 31.1 #7.46, infected with the vector/TMT construct were both TMT-negative implying that transformation efficiency in this species was not 100%. More importantly though, is that both clones selected visually were indeed TMT-positive.

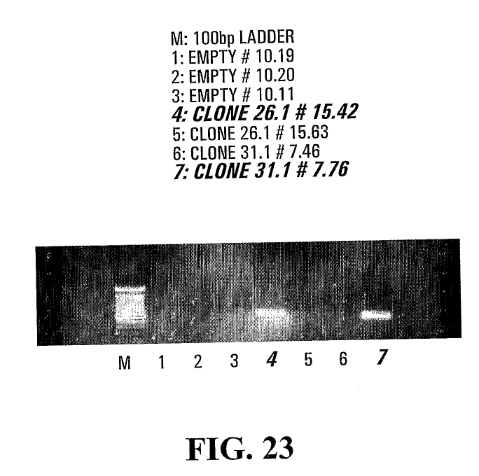

With reference to FIG. 23, PCR using TMT-specific primers showed that both basil root clones chosen on the basis of length (see FIG. 22) did possess the TMT gene demonstrating that the iodide methylation into CH₃I was indeed due to the TMT gene introduced in those clones. All the lanes where no product was obtained indeed did not show any TMT activity (see FIG. 22).

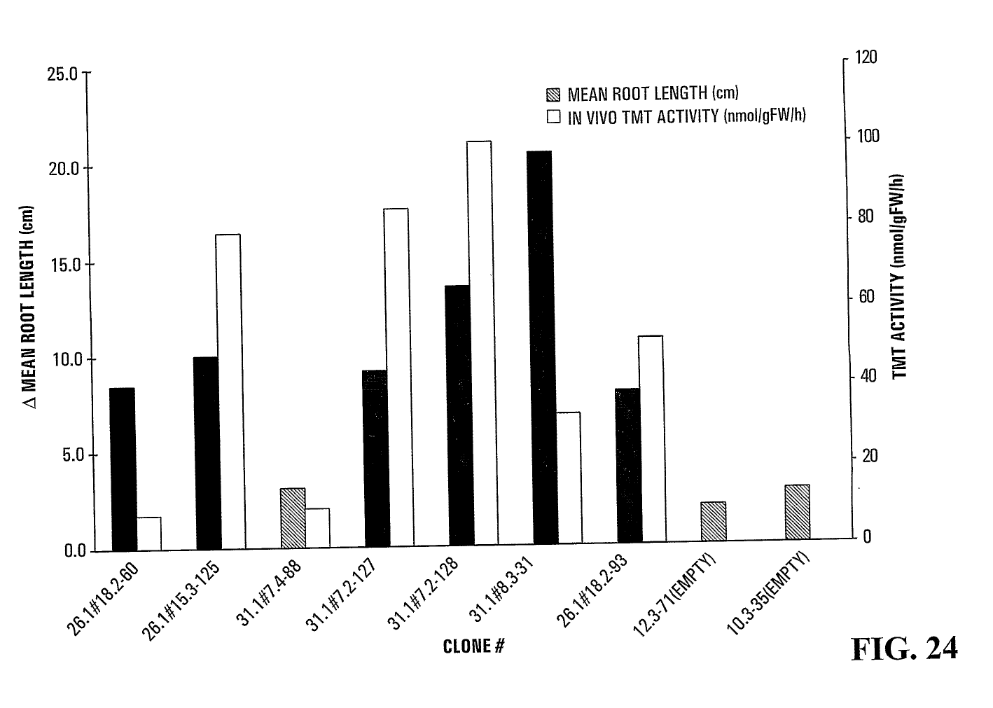

With reference to FIG. 24, all 6 potato root clones chosen on the basis of length (solid-filled bars) turned out to be TMT-positive. Transformation efficiency was 100% since clones 31.1 #7.4-88 which was infected with the vector/TMT, also turned out to be TMT-positive although it was not visually selected. Therefore, clones that were deemed positive chosen upon visual examination were all TMT-positive.

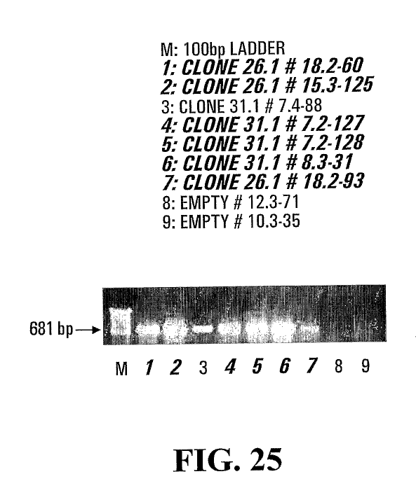

With reference to FIG. 25, PCR using TMT-specific primers showed that all 6 potato root clones chosen on the basis of length (see FIG. 24) did possess the TMT gene demonstrating that the iodide methylation into CH₃I was indeed due to the TMT gene introduced in those clones. Clone 31.1 #7.4-88 (lane 3) gave a PCR product proving that the TMT activity obtained from this clone was indeed from the TMT gene introduced (this clone was, however, not visually selected). Both the empty clones (lanes 8-9) did not give a PCR product and did not show any TMT activity (see FIG. 24).

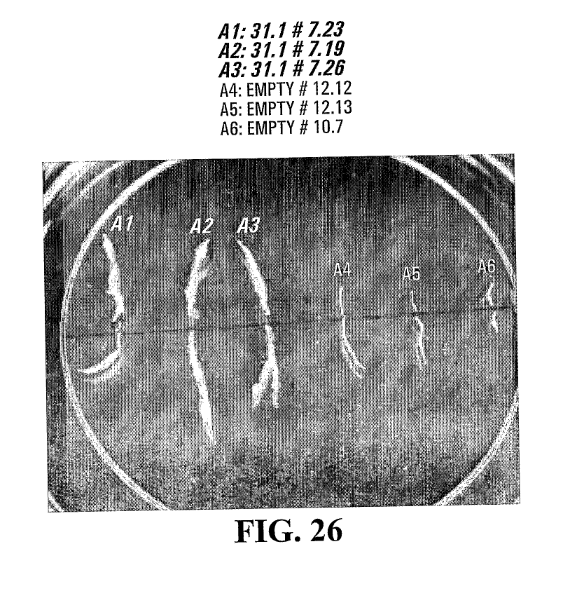

With reference to FIG. 26, the Petri dish shown contains 7.5 mM SCN and the roots are the individual green bean clones randomly chosen for assay. From this assay, root clones A1, A2 and A3 were selected as putative positive based solely on their greater length. All 3 clones chosen visually were shown to be positive for TMT activity (see FIG. 25).

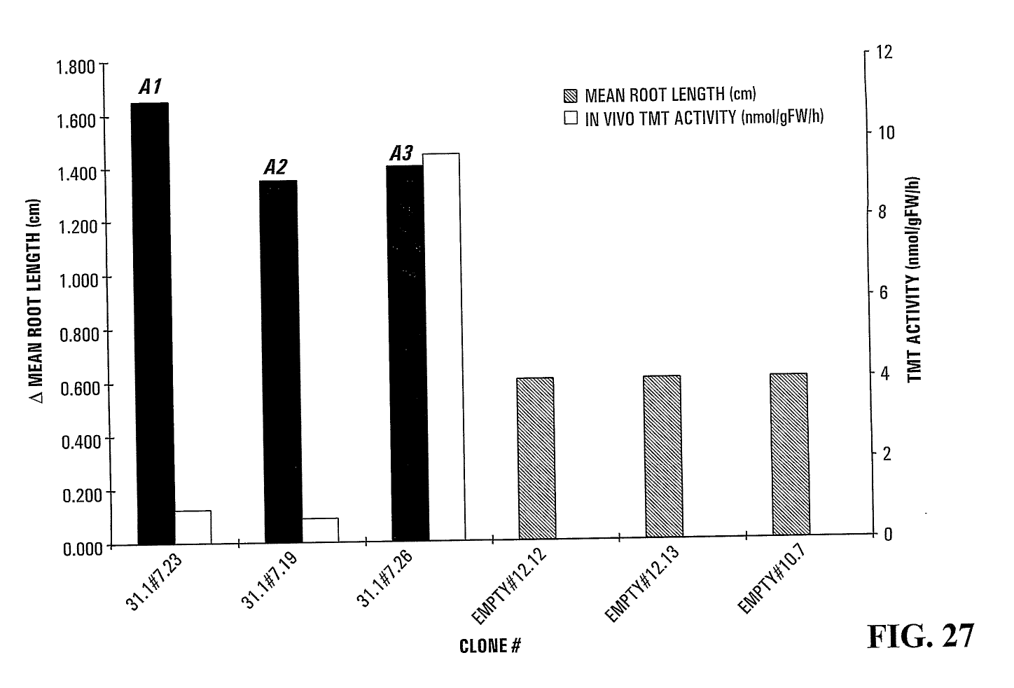

With reference to FIG. 27, all 3 green bean clones chosen on the basis of length (solid-filled bars) were found to be TMT-positive. Selection efficiency was, therefore, 100%.

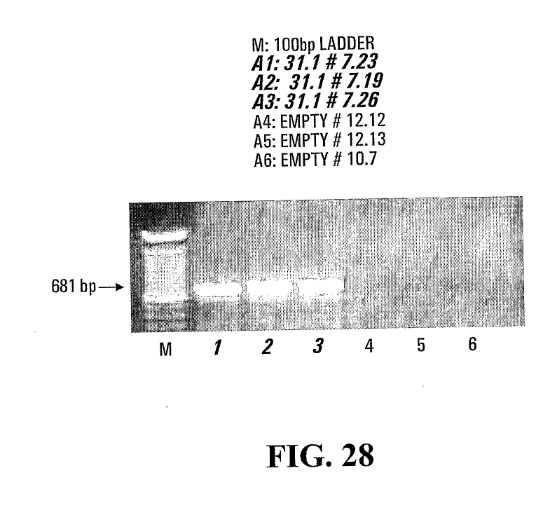

With reference to FIG. 28, PCR using TMT-specific primers showed that all 3 green bean clones chosen on the basis of length (see FIGS. 26 and 27) did possess the TMT gene proving that the iodide methylation into CH₃I was indeed due to the TMT gene introduced in those clones. All 3 empty clones (lanes 4-6) did not give a PCR product and did not show any TMT activity (see FIG. 27).

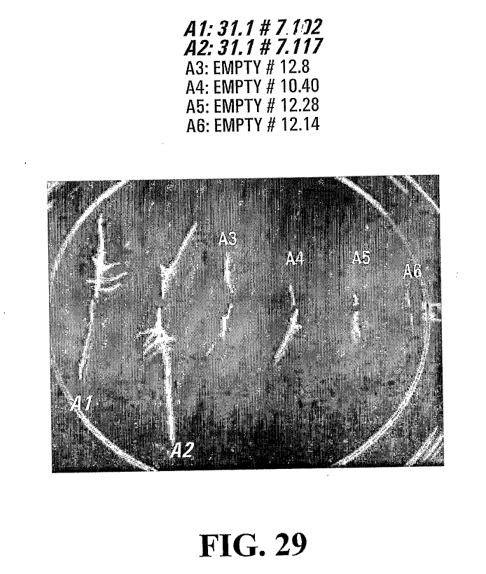

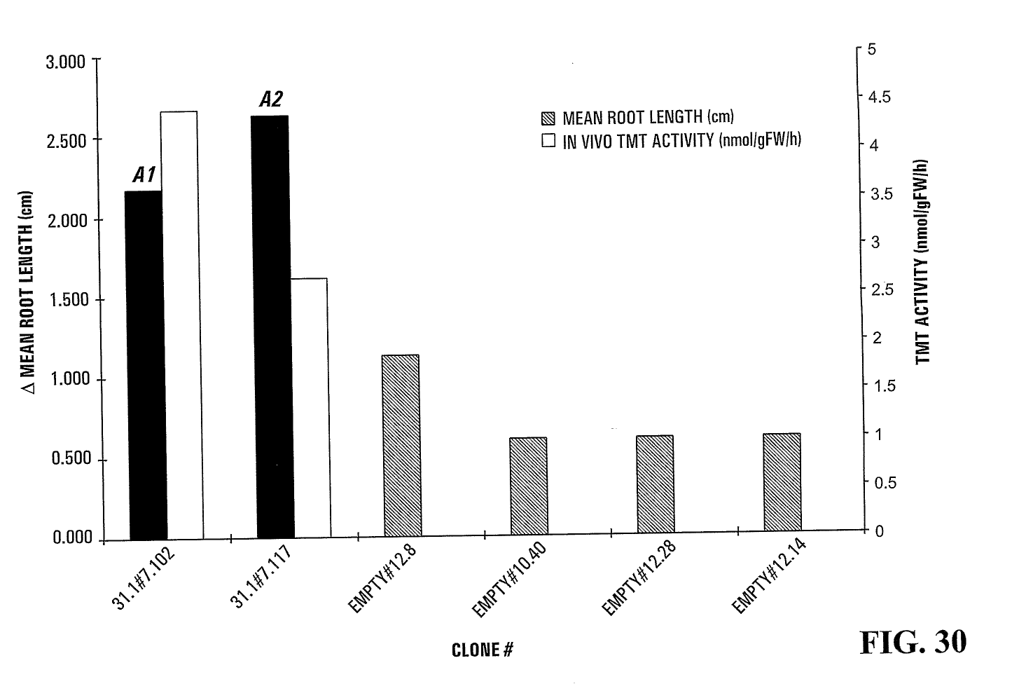

With reference to FIG. 29, from this assay, tomato root clones A1 and A2 were selected as putative positive based solely on their greater length. Both clones chosen visually turned out to be positive for TMT activity (see FIG. 30).

With reference to FIG. 30, both tomato root clones chosen on the basis of length (solid-filled bars) turned out to be TMT-positive. Selection efficiency was, therefore, 100%.

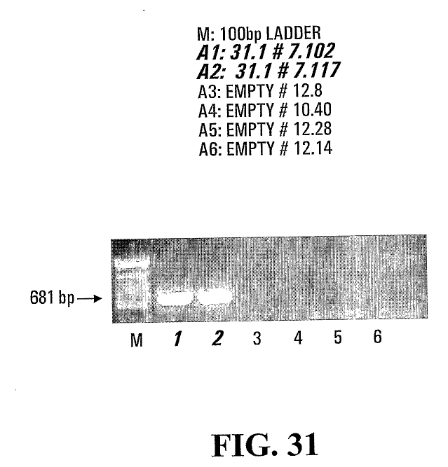

With reference to FIG. 31, PCR using TMT-specific primers showed that both tomato root clones chosen on the basis of length (see FIGS. 29 and 30) did possess the TMT gene proving that the iodide methylation into CH₃I was indeed due to the TMT gene introduced in those clones. All 4 empty clones (lanes 3-6) did not give a PCR product and did not show any TMT activity (see FIG. 30).

### Example 6

### Use of TMT as a Selectable Marker in Whole Dicot Plants

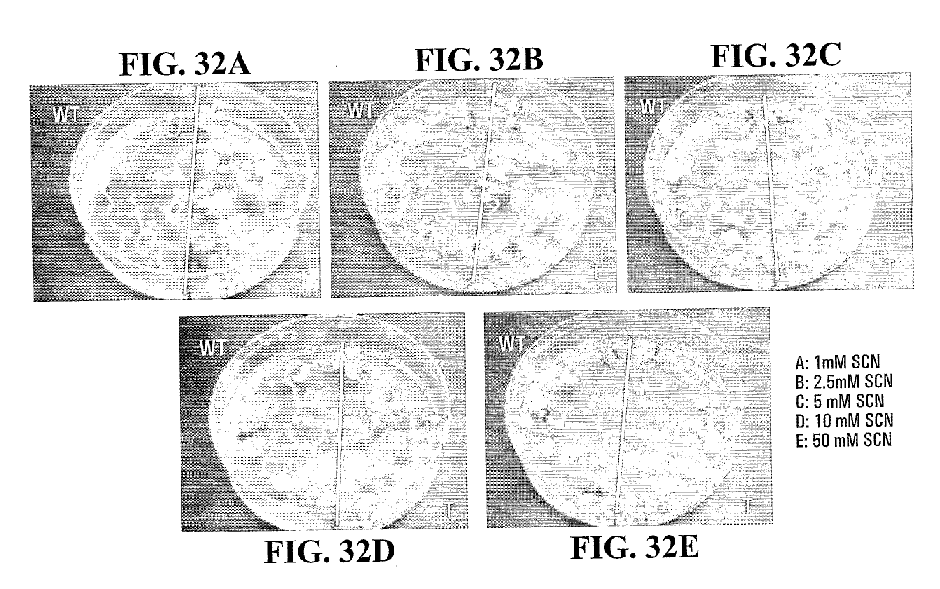

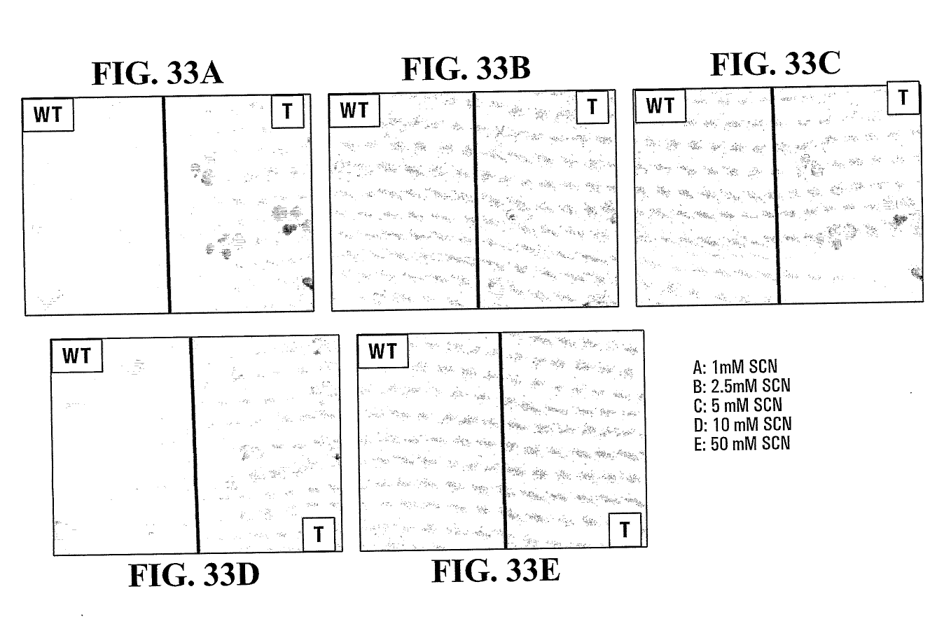

With reference to FIG. 32, seeds of wild type (WT) and TMT transgenic (T) tobacco plants (produced by transformation using conventional A. tumefaciens infection and selected with kanamycin) were sown in MS/phytagel containing different SCN concentrations. Pictures were taken 21 days after germination. It can be clearly seen that at all SCN concentrations (except 50 mM which was lethal to both phenotypes), that the transgenic tobacco plants (T) grew better than the wild type plants which lack TMT, indicating that TMT may be used as a selectable marker in tobacco using SCN as a potential selective agent. FIG. 33 is a higher magnification image of FIG. 32.

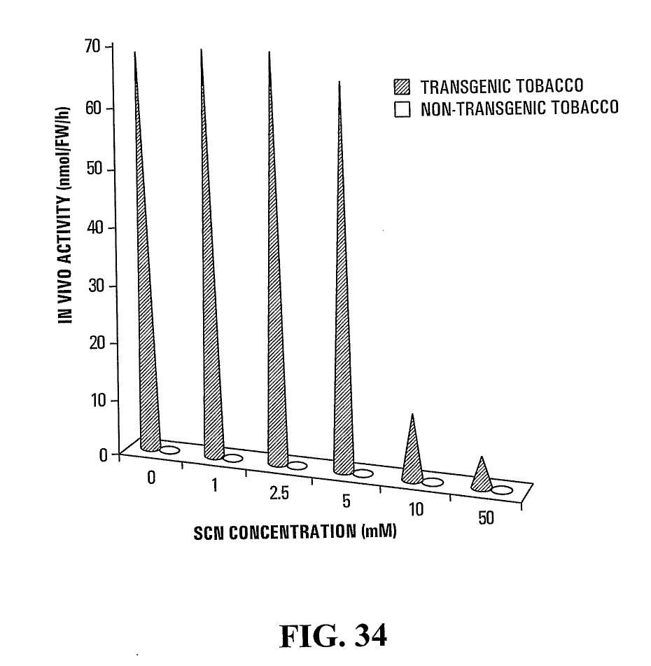

With reference to FIG. 34, the effect of SCN on in vivo TMT activity of transgenic/non-transgenic tobacco plants was analysed. Consistent with the results of FIG. 33, in vivo TMT activity confirms that the transgenic tobacco indeed had a positive activity while the non-transgenic plant did not have any activity at any SCN concentration tested. As the concentration of SCN increased to 5 mM, the transgenic plant was able to methylate the SCN but beyond 5 mM, the activity decreased as higher SCN levels appear to confer some toxicity to the plant.

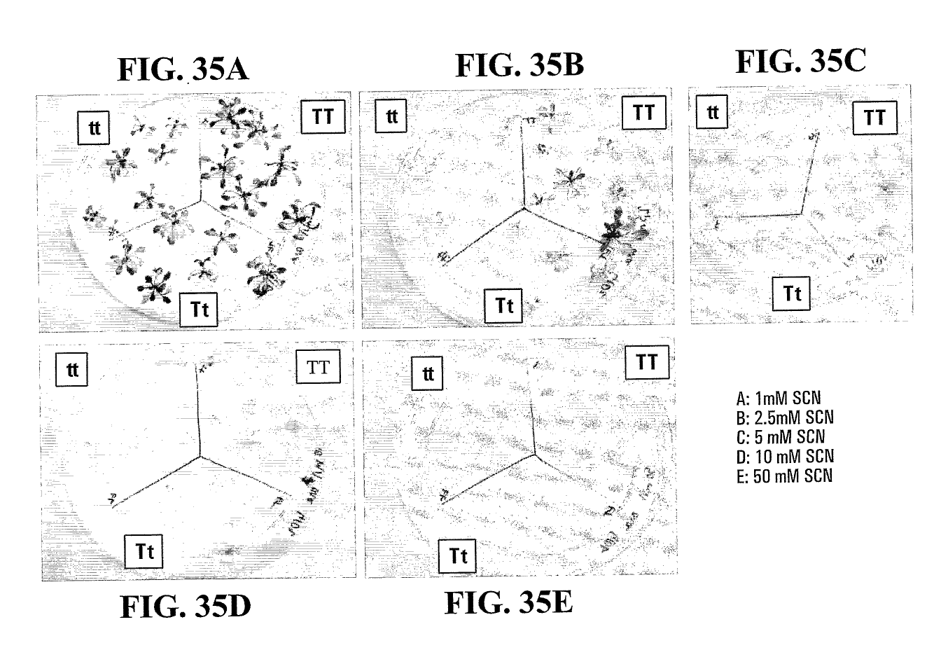

With reference to FIG. 35, wild type (TT) (naturally contains TMT), heterozygous (Tt) and double recessive (tt) TMT knock out A. thaliana plants were sown in MS/phytagel containing different SCN concentrations. Pictures were taken 21 days after germination. It can be clearly seen that at all SCN concentrations (except 50 mM which was lethal to all 3 phenotypes), TT grew better than the Tt which in turn grew better than tt. This indicates that TMT may be used as a selectable marker in A. thaliana using SCN as a potential selective agent.

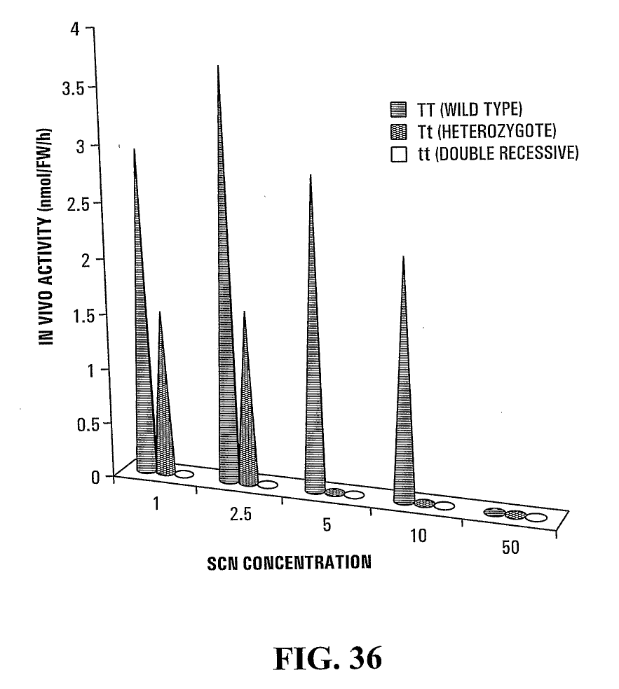

With reference to FIG. 36, the effect of SCN on TMT activity in 3 Arabidopsis phenotypes was analysed. Consistent with the results of FIG. 35, in vivo TMT activity shows that the tt plants (double recessive TMT knock-out) did not have any TMT activity while the two other phenotypes (TT and Tt) were positive for TMT activity. As the concentration of SCN increased the TT and Tt plants were less and less able to methylate the SCN. TT plants could methylate the SCN up to 10 mM while the Tt had no activity after 2.5 mM, as it appears that higher levels of SCN may have some toxicity to the heterozygote plant.

Throughout this application, various references are cited, which describe more fully the state of the art to which this invention pertains. The disclosures of these references are hereby incorporated by reference into the present disclosure.

1. A method for selecting a first cell from a population of cells, said method comprising:
(a) providing said population of cells comprising said first cell, wherein said first cell comprises a first nucleic acid which encodes a polypeptide having TMT activity;
(b) contacting said population of cells with a toxin, wherein said toxin is a TMT substrate; and
(c) selecting said first cell by virtue of its increased tolerance to said toxin.

2. The method of claim 1, wherein said first cell comprises a recombinant vector comprising said first nucleic acid.

3. The method of claim 2, wherein step (a) comprises transforming a population of cells with said recombinant vector thereby to provide said population of cells comprising said first cell.

4. The method of claim 1, wherein the toxin is a halide-containing compound.

5. (canceled)

6. The method of claim 1, wherein the toxin is a sulfur-containing compound.

7. The method of claim 6, wherein the sulfur-containing compound is selected from the group consisting of a thiocyanate, a sulfide, a thiol and a thiolate.

8-16. (canceled)

17. The method of claim 1, wherein the first cell is a prokaryotic or an eukaryotic cell.

18. The method of claim 17, wherein the eukaryotic cell is a plant cell or an animal cell.

19-36. (canceled)

37. The method of claim 1, wherein said polypeptide is a plant TMT.

38. The method of claim 37, wherein said plant TMT is a Brassicaceae TMT.

39. The method of claim 38, wherein the Brassicaceae TMT is a Brassica TMT.

40. (canceled)

41. The method of claim 40, wherein said Brassica oleracea TMT comprises a polypeptide having a sequence substantially identical to a sequence selected from the group consisting of SEQ ID NO: 2, SEQ ID NO: 4 and SEQ ID NO: 6.

42-44. (canceled)

45. A method for selecting a first plant or part thereof from a population of plants or parts thereof, said method comprising:
(a) providing said population of plants or parts thereof comprising said first plant or part thereof, wherein said first plant or part thereof comprises a first nucleic acid which encodes a polypeptide having TMT activity;
(b) contacting said population of plants or parts thereof with a toxin, wherein said toxin is a TMT substrate; and
(c) selecting said first plant or part thereof by virtue of its increased tolerance to said toxin.

46-50. (canceled)

51. The method of claim 45, wherein said first plant or part thereof comprises a recombinant vector comprising said first nucleic acid.

52. A plant cell comprising a recombinant vector comprising a first nucleic acid which encodes a polypeptide having TMT activity.

53. The plant cell of claim 52, wherein said polypeptide is a plant TMT.

54-64. (canceled)

65. A transgenic plant comprising the plant cell of claim 52, or a progeny, tissue, organ or seed thereof.

66. A method of preparing the transgenic plant of claim 65, said method comprising regenerating the transgenic plant from the plant cell of claim 52.

67. A selectable marker system for the selection of a first cell from a population of cells comprising:
(a) a recombinant vector comprising a first nucleic acid which encodes a polypeptide having TMT activity; and
(b) a toxin, wherein said toxin is a TMT substrate.

68-70. (canceled)

71. A commercial package comprising:
(a) a recombinant vector comprising a nucleic acid encoding a polypeptide having TMT activity; and
(b) instructions for the selection of a first cell from a population of cells.

72-76. (canceled)
